(19) United States
(12) Patent Application Publication
(10) Pub. No.: US 2014/0051696 A1
(43) Pub. Date: Feb. 20, 2014

(54) THERAPIES FOR TREATING CANCER

(71) Applicant: GILEAD CALISTOGA LLC, Foster City, CA (US)

(72) Inventors: Brian J. LANNUTTI, Seattle, WA (US); Julie DI PAOLO, Northford, CT (US); Heather WEBB, Seattle, WA (US); Adam KASHISHIAN, Edmonds, WA (US)

(73) Assignee: GILEAD CALISTOGA LLC, Foster City, CA (US)

(21) Appl. No.: 13/967,313

(22) Filed: Aug. 14, 2013

Related U.S. Application Data
(60) Provisional application No. 61/683,191, filed on Aug. 14, 2012.
(60) Provisional application No. 61/724,870, filed on Nov. 9, 2012.
(60) Provisional application No. 61/800,853, filed on Mar. 15, 2013.

(51) Int. Cl. A61K 31/5377; A61K 31/52

(52) U.S. Cl. CPC A61K 31/5377; A61K 31/52
USPC 514/233.2

(57) ABSTRACT

Provided herein are methods that relate to a novel therapeutic strategy for treatment of cancer, including hematological malignancies such as leukemia, lymphoma, and multiple myeloma. In particular, the method includes administration of a PI3Kδ inhibitor and a Syk inhibitor.

### CROSS REFERENCE TO RELATED APPLICATIONS

This application claims the benefit of U.S. provisional patent application Ser. Nos. 61/683,191, filed Aug. 14, 2012, 61/724,870, filed Nov. 9, 2012, and 61/800,853, filed Mar. 15, 2013, the disclosures of which are hereby incorporated herein by reference in their entireties.

### FIELD

The present disclosure relates generally to the field of therapeutics and medicinal chemistry, and more specifically to the treatment of cancer including, for example, leukemia, lymphoma, and multiple myeloma.

### BACKGROUND

Cancer generally remains incurable with standard therapies. One example of such a cancer is chronic lymphocytic leukemia (CLL), a neoplasm resulting from the progressive accumulation of functionally incompetent monoclonal B lymphocytes in blood, bone marrow, lymph nodes, spleen, and liver.

In younger and relatively healthy patients with CLL, chemoimmunotherapy regimens that include the anti-CD20 monoclonal antibody, rituximab, are commonly employed to control disease manifestations. See Gribben & O'Brien, J. Clin. Oncol. 2011; 29 (5):544-50. However, in elderly patients or patients with comorbid conditions, such regimens are associated with less efficacy and greater toxicity and increasing attention has been paid to the problem of treating patients with CLL who have comorbidities. See Tam et al., Br. J. Haematol. 2008; 141 (1):36-40; Eichhorst et al., Leuk. Lymphoma, 2009; 50 (2):171-8; and Goede & Hallek, Drugs Aging 2011; 28 (3):163-76. Because of the relatively late age of diagnosis, a large proportion (˜90%) of patients with CLL have comorbidities and a substantial proportion (˜45%) have major chronic conditions such as coronary artery disease, diabetes, or chronic obstructive pulmonary disease. At the time the disease is first identified, ˜25% of patients with CLL do not meet conventional criteria for participation in clinical studies containing cytotoxic agents. See Thurmes et al., Leuk. Lymphoma 2008; 49 (1):49-56.

These health constraints in older or compromised patients have prompted noncytotoxic approaches to therapy. Alternative immunotherapeutics, such as the monoclonal antibodies, alemtuzumab or ofatumumab have been developed. See Keating et al., Blood 2002; 99 (10):3554-61; and Wierda et al., J. Clin. Oncol. 2010; 28 (10):1749-55. However the therapeutic utility of the two drugs is modest; median progression-free survival (PFS) values in patients with recurrent CLL have been 4.7 months and 5.8 months, respectively. Moreover, these treatments can lead to other issues. For example, alemtuzumab can cause extreme immunosuppression that can lead to frequent opportunistic infection. Administration of the large amounts of protein recommended in product labeling for ofatumumab results in frequent infusion reactions and cumbersome infusion schedules.

In view of these conditions, repeated use of rituximab monotherapy or rituximab-corticosteroid combinations have been advocated in treatment guidelines for older or frail patients with recurrent CLL. See Eichhorst et al., Ann. Oncol. 2010; 21 Suppl 5:v162-4; and Zelenetz et al., J. Natl. Compr. Canc. Netw. 2011; 9 (5):484-560. While single-agent rituximab use can offer certain benefit such as good tolerability in some patients with previously treated CLL, tumor control is not lasting, especially in patients with bulky adenopathy. See Gentile et al., Cancer management and research 2010; 2:71-81. Addition of high-dose methylprednisone to rituximab can extend median PFS to 12 months, but this combination is commonly associated with severe hyperglycemia and frequent life-threatening or fatal infections. See e.g., Bowen et al., Leuk Lymphoma 2007; 48 (12):2412-7; and Dungarwalla et al., Haematologica 2008; 93 (3):475-6.

As such, new noncytotoxic, well-tolerated, and convenient therapies are needed in order to enhance and prolong tumor control in patients with comorbid conditions. Due to the limitations of current treatments for cancer, there remains a significant interest in and need for additional or alternative therapies for treating, stabilizing, preventing, and/or delaying cancer.

### SUMMARY

Provided herein are methods, compositions, articles of manufacture, and kits for treating a cancer by using effective amounts of two or more inhibitors selected from a PI3Kδ inhibitor, a Syk inhibitor, and a Btk inhibitor.

In one aspect, provided is a method for treating a subject (e.g., a human), who has or is suspected of having a cancer, by administering to the subject in need of such treatment an effective amount of Compound A or Compound D

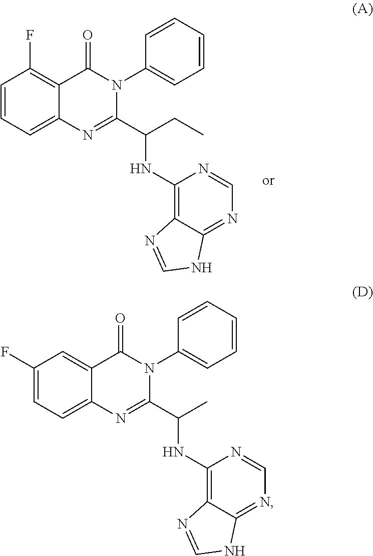

or a pharmaceutically acceptable salt, prodrug, or solvate thereof, and an effective amount of Compound B or Compound C

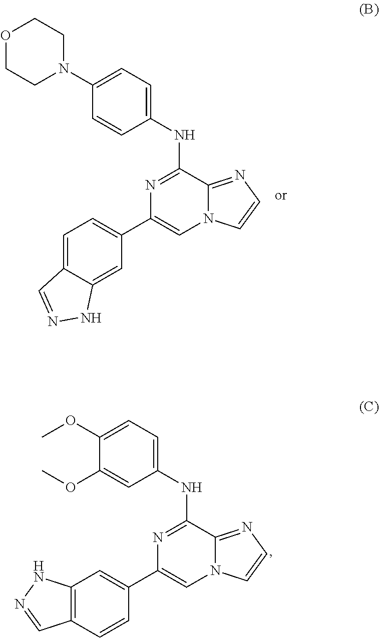 [SMILES: c1cn2cc(-c3ccc4cn[nH]c4c3)nc(Nc3ccc(N4CCOCC4)cc3)c2n1 ; COc1ccc(Nc2nc(-c3ccc4cn[nH]c4c3)cn3ccnc23)cc1OC]

or a pharmaceutically acceptable salt, prodrug, or solvate thereof.

In certain embodiments, an effective amount of Compound A or a pharmaceutically acceptable salt, prodrug, or solvate thereof and an effective amount of Compound B or a pharmaceutically acceptable salt, prodrug, or solvate thereof are administered to the subject in need of such treatment. In other embodiments, an effective amount of Compound D or a pharmaceutically acceptable salt, prodrug, or solvate thereof and an effective amount of Compound B or a pharmaceutically acceptable salt, prodrug, or solvate thereof are administered to the subject in need of such treatment.

In one embodiment, Compound A or Compound D, or a pharmaceutically acceptable salt, prodrug, or solvate thereof is predominantly the S-enantiomer.

In some embodiments, Compound A or Compound D or a pharmaceutically acceptable salt, prodrug, or solvate thereof is present in a pharmaceutical composition that includes Compound A or Compound D or a pharmaceutically acceptable salt, prodrug, or solvate thereof, and at least one pharmaceutically acceptable vehicle. In some embodiments, Compound B or a pharmaceutically acceptable salt, prodrug, or solvate thereof is present in a pharmaceutical composition that includes Compound B or a pharmaceutically acceptable salt, prodrug, or solvate thereof, and at least one pharmaceutically acceptable vehicle. In yet other embodiments, Compound A and Compound B, or Compound D and Compound B, or pharmaceutically acceptable salts, prodrugs, or solvates thereof, are both present in a pharmaceutical composition that includes Compound A or Compound D, Compound B, or pharmaceutically acceptable salts, prodrugs, or solvates thereof, and at least one pharmaceutically acceptable vehicle.

In some embodiments, Compound B or a pharmaceutically acceptable salt thereof is administered before Compound A or a pharmaceutically acceptable salt thereof. In other embodiments, Compound A or Compound D, or a pharmaceutically acceptable salt thereof, and Compound B or a pharmaceutically acceptable salt thereof, are administered simultaneously. In certain embodiments, each of Compound A and Compound B, or each of Compound D and Compound B, or pharmaceutically acceptable salts thereof is independently administered once a day or twice a day.

In some embodiments, Compound A or Compound D or a pharmaceutically acceptable salt thereof is administered at a dose between 50 mg and 200 mg; and Compound B or a pharmaceutically acceptable salt thereof is administered at a dose between 100 mg and 750 mg. In certain embodiments, the dose of Compound A or Compound D or a pharmaceutically acceptable salt thereof is administered as one or more unit dosages each independently comprising between 50 mg and 200 mg of Compound A or Compound D or a pharmaceutically acceptable salt thereof; and the dose of Compound B or a pharmaceutically acceptable salt thereof is administered as one or more unit dosages each independently comprising between 100 mg and 300 mg of Compound B or a pharmaceutically acceptable salt thereof. In one embodiment, the dose of Compound A or Compound D or a pharmaceutically acceptable salt thereof is 100 mg or 150 mg; and the dose of Compound B or a pharmaceutically acceptable salt thereof is 200 mg or 600 mg. In yet another embodiment, the dose of Compound A or Compound D or a pharmaceutically acceptable salt thereof is administered as a unit dosage comprising 100 mg or 150 mg of Compound A or Compound D or a pharmaceutically acceptable salt thereof; and the dose of Compound B or a pharmaceutically acceptable salt thereof is administered as one or more unit dosages each independently comprising 25 mg, 100 mg or 200 mg of Compound B or a pharmaceutically acceptable salt thereof. In one embodiment, the unit dosage is a tablet.

In some embodiments, Compound A and Compound B, or pharmaceutically acceptable salts thereof, are administered under fed conditions. In other embodiments, Compound D and Compound B, or pharmaceutically acceptable salts thereof, are administered under fed conditions.

In other embodiments, the subject who has cancer is (i) refractory to at least one chemotherapy treatment, or (ii) is in relapse after treatment with chemotherapy, or a combination thereof. In certain embodiments, the subject has not previously been treated for the cancer. In one embodiment, the subject is a human subject.

In some embodiments, the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL). In certain embodiments, the cancer is leukemia, lymphoma, or multiple myeloma. In certain embodiments, the cancer is Burkitt's lymphoma, Hodgkin's lymphoma, non-Hodgkin's lymphoma, lymphocytic lymphoma, lymphocytic leukemia, multiple myeloma, or chronic myeloid leukemia. In one embodiment, the cancer chronic lymphocytic leukemia, B-cell acute lymphocytic leukemia, diffuse large B-cell lymphoma, or mantle cell lymphoma. In one embodiment, the cancer is minimal residual disease (MRD).

In particular embodiments, the cancer is leukemia or lymphoma. In specific embodiments, the cancer is acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), multiple myeloma (MM), indolent non-Hodgkin's lymphoma (iNHL), refractory iNHL, non-Hodgkin's lymphoma (NHL), mantle cell lymphoma (MCL), follicular lymphoma, Waldestrom's macroglobulinemia (WM), T-cell lymphoma, B-cell lymphoma, and diffuse large B-cell lymphoma (DLBCL). In one embodiment, the cancer is T-cell acute lymphoblastic leukemia (T-ALL), or B-cell acute lymphoblastic leukemia (B-ALL). The non-Hodgkin lymphoma encompasses the indolent B-cell diseases that include, for example, follicular lymphoma, lymphoplasmacytic lymphoma, Waldenstrom macroglobulinemia, and marginal zone lymphoma, as well as the aggressive lymphomas that include, for example, Burkitt lymphoma, diffuse large B-cell lymphoma (DLBCL) and mantle cell lymphoma (MCL). In one embodiment, the leukemia is minimal residual disease (MRD).

Provided is also a method for decreasing cell viability in cancer cells in a human, comprising administering to the human Compound A or Compound D or a pharmaceutically acceptable salt thereof, and Compound B or a pharmaceutically acceptable salt thereof in amounts sufficient to detectably decrease cell viability in the cancer cells. Provided is also a method for decreasing cell viability in cancer cells, comprising contacting cancer cells with Compound A or Compound D or a pharmaceutically acceptable salt thereof, and Compound B or a pharmaceutically acceptable salt thereof in amounts sufficient to detectably decrease cell viability in the cancer cells. In some embodiments, the cell viability in the cancer cells after administering to the human, or contacting the cancer cells with, Compound A and Compound B, or with Compound D and Compound B or pharmaceutically acceptable salts thereof, is decreased by at least 10% compared to cell viability in cancer cells after administering to the human, or contacting the cancer cells with, only Compound A or Compound D, or a pharmaceutically acceptable salt thereof or after administering to the human, or contacting the cancer cells with, only Compound B, or a pharmaceutically acceptable salt thereof. In one embodiment, cell viability in the cancer cells is determined by a cell viability assay, such as MTS assay.

Provided is also a method for decreasing AKT phosphorylation, S6 phosphorylation, or AKT and S6 phosphorylation in cancer cells in a human, comprising administering to the human Compound A or C or a pharmaceutically acceptable salt thereof, and Compound B or a pharmaceutically acceptable salt thereof in amounts sufficient to detectably decrease AKT phosphorylation, S6 phosphorylation, or AKT and S6 phosphorylation in the cancer cells. Provided is also a method for decreasing AKT phosphorylation, S6 phosphorylation, or AKT and S6 phosphorylation in cancer cells, comprising contacting cancer cells with Compound A or C or a pharmaceutically acceptable salt thereof, and Compound B or a pharmaceutically acceptable salt thereof in amounts sufficient to detectably decrease AKT phosphorylation, S6 phosphorylation, or AKT and S6 phosphorylation in the cancer cells. In some embodiments, S6 phosphorylation in the cancer cells after administering to the human, or contacting the cancer cells with, Compound A and Compound B, or with Compound D and Compound B, or pharmaceutically acceptable salts thereof, is decreased by at least 10% compared to S6 phosphorylation in cancer cells after administering to the human, or contacting the cancer cells with, only Compound A or Compound D, or a pharmaceutically acceptable salt thereof or after administering to the human, or contacting the cancer cells with, only Compound B, or a pharmaceutically acceptable salt thereof. In one embodiment, AKT phosphorylation, S6 phosphorylation, or AKT and S6 phosphorylation in the cancer cells is/are determined by flow cytometry. In certain embodiments, the cancer cells are chronic lymphocytic leukemia (CLL) cells.

In another aspect, provided is a method for decreasing AKT phosphorylation, ERK phosphorylation, or AKT and ERK phosphorylation in cancer cells in a human, comprising administering to the human Compound A or Compound D or a pharmaceutically acceptable salt thereof, and Compound B or a pharmaceutically acceptable salt thereof in amounts sufficient to detectably decrease AKT phosphorylation, ERK phosphorylation, or AKT and ERK phosphorylation in the cancer cells. In another aspect, provided is a method for decreasing AKT phosphorylation, ERK phosphorylation, or AKT and ERK phosphorylation in cancer cells, comprising contacting cancer cells with Compound A or Compound D or a pharmaceutically acceptable salt thereof, and Compound B or a pharmaceutically acceptable salt thereof in amounts sufficient to detectably decrease AKT phosphorylation, ERK phosphorylation, or AKT and ERK phosphorylation in the cancer cells. In some embodiments, ERK phosphorylation in the cancer cells after administering to the human, or contacting the cancer cells with, Compound A and Compound B, or with Compound D and Compound B or pharmaceutically acceptable salts thereof, is decreased by at least 10% compared to ERK phosphorylation in cancer cells after administering to the human, or contacting the cancer cells with, only Compound A or Compound D, or a pharmaceutically acceptable salt thereof or after administering to the human, or contacting the cancer cells with, only Compound B, or a pharmaceutically acceptable salt thereof. In some embodiments, AKT phosphorylation, ERK phosphorylation, or AKT and ERK phosphorylation in the cancer cells is/are determined by immunoblotting. In one embodiment, the cancer cells are Burkitt's lymphoma cells.

In yet another aspect, provided is a method of decreasing chemokine production in a sample comprising cells expressing CCL2, CCL3, CCL4, CCL22, or any combinations thereof, comprising contacting the sample with Compound A or Compound D or a pharmaceutically acceptable salt thereof, and Compound B or a pharmaceutically acceptable salt thereof in amounts sufficient to detectably chemokine production in the sample. In some embodiments, one or more of the following (i)-(iv) applies: (i) CLL2 production in the cells after contact with Compound A and Compound B, or with Compound D and Compound B, or pharmaceutically acceptable salts thereof, is decreased by at least 5% compared to CLL2 production in the cells after contact with only Compound A or Compound D, or a pharmaceutically acceptable salt thereof or after contact with only Compound B, or a pharmaceutically acceptable salt thereof; (ii) CLL3 production in the cells after contact with Compound A and Compound B, with Compound D and Compound B, or pharmaceutically acceptable salts thereof, is decreased by at least 5% compared to CLL3 production in the cells after contact with only Compound A or Compound D, or a pharmaceutically acceptable salt thereof or after contact with only Compound B, or a pharmaceutically acceptable salt thereof; (iii) CLL4 production in the cells after contact with Compound A and Compound B, or with Compound D and Compound B, or pharmaceutically acceptable salts thereof, is decreased by at least 5% compared to CLL4 production in the cells after contact with only Compound A or Compound D, or a pharmaceutically acceptable salt thereof or after contact with only Compound B, or a pharmaceutically acceptable salt thereof; and (iv) CLL22 production in the cells after contact with Compound A and Compound B, or with Compound D and Compound B, or pharmaceutically acceptable salts thereof, is decreased by at least 5% compared to CLL22 production after contact with only Compound A or Compound D, or a pharmaceutically acceptable salt thereof or after contact with only Compound B, or a pharmaceutically acceptable salt thereof. In one embodiment, the chemokine production in the sample is determined by an immunoassay.

In any of the foregoing embodiments related to the method for decreasing cell viability, decreasing AKT phosphorylation, S6 phosphorylation, or AKT and S6 phosphorylation, decreasing AKT phosphorylation, ERK phosphorylation, or AKT and ERK phosphorylation, and decreasing chemokine production in cells, the method may be performed in vitro, in vivo, or ex vivo. When the method is performed in vivo, in one aspect, the method comprises administering Compound A and Compound B, or Compound D and Compound B, to an a subject (e.g., a human) in need thereof.

In yet another aspect, provided is a kit comprising: (i) a pharmaceutical composition comprising Compound A or Compound D or a pharmaceutically acceptable salt thereof, and at least one pharmaceutically acceptable vehicle; and (ii) a pharmaceutical composition comprising Compound B or a pharmaceutically acceptable salt thereof, and at least one pharmaceutically acceptable vehicle. In some embodiments, the method further comprises: a package insert containing instructions for use of the pharmaceutical compositions in treating a cancer. In one embodiment, each pharmaceutical composition is independently a tablet.

Provided herein is also an article of manufacture comprising: (i) a unit dosage form of Compound A or Compound D or a pharmaceutically acceptable salt thereof, and at least one pharmaceutically acceptable vehicle; (ii) a unit dosage form of Compound B or a pharmaceutically acceptable salt thereof; and at least one pharmaceutically acceptable vehicle; and (iii) a label containing instructions for use of Compound A and Compound B, or for use of Compound D and Compound B, or pharmaceutically acceptable salts thereof, in treating cancer. In some embodiments, each unit dosage form is a tablet.

In another aspect, provided is a method of sensitizing cancer cells in a human receiving a treatment of Compound A or Compound D or a pharmaceutically acceptable salt thereof, wherein the method comprises administering to the human Compound B or a pharmaceutically acceptable salt thereof before or concurrently with treating the human with Compound A or Compound D, or a pharmaceutically acceptable salt thereof. In another aspect, provided is a method of sensitizing cancer cells receiving a treatment of Compound A or Compound D or a pharmaceutically acceptable salt thereof, wherein the method comprises contacting the cancer cells with Compound B or a pharmaceutically acceptable salt thereof before or concurrently with treating the cancer cells with Compound A or Compound D, or a pharmaceutically acceptable salt thereof.

In yet another aspect, provided is a method of sensitizing a subject who is (i) substantially refractory to at least one chemotherapy treatment, or (ii) is in relapse after treatment with chemotherapy, or both (i) and (ii), wherein the method comprises administering to the subject an effective amount of Compound A or Compound D or a pharmaceutically acceptable salt thereof, and an effective amount of Compound B or a pharmaceutically acceptable salt thereof.

### DESCRIPTION OF THE FIGURES

The present application can be best understood by references to the following description taken in conjunction with the accompanying figures.

FIG. 1 depicts a graph showing inhibition of CD63 expression on human basophils by administering Compound B and Compound A in combination.

FIG. 2 depicts a graph showing inhibition of pMek in DHL-4 cells by administering Compound B and Compound A in combination.

FIG. 3 depicts a graph showing in vitro inhibition of CCL4 release from CCRF-SB cells by administering Compound B and Compound A in combination.

FIGS. 4A, B, E-O depict drug response curves for peripheral blood samples. FIGS. 4C, P-R depict drug response curves for bone marrow samples. In particular, FIGS. 4A and 4B depict drug response curves for an individual sample: multiple synergistic points (FIG. 4A) and additive response (FIG. 4B). FIG. 4C depicts a drug response curve for a sample of CLL cells isolated from bone marrow. FIG. 4D depicts a graph showing the interaction indices for each sample tested, where an interaction index of <1 indicates synergy. Asterisks (*) shows synergistic concentrations within a 95% confidential interval.

FIGS. 5A and 5B depict graphs showing inhibition of AKT and S6 phosphorylation, respectively.

FIG. 6 depicts immunoblot AKT and ERK expression profiles after culturing of Ramos cells with Compound A and Compound B, alone or in combination, for 24 hours.

FIGS. 7A-D depicts graphs showing the effect of Compound A and/or Compound B on CCL2, CCL3, CCL4 and CCL22 secretion after CLL-HS-5 co-culture.

FIG. 7E depicts a graph showing the effect of Compound B and Compound A on CCL4 chemokine release in CCRF-SB cell line.

FIGS. 7F-I depicts graphs showing the effects of Compound A and Compound B on CCL2, CCL3, CCL4, and CCL22 secretion after treatment of CLL cells cultured in patient matched primary BM stromal cells. Asterisks (*) denote significant (p<0.05) differences between single agent treatment and the combinations as shown.

FIGS. 8A and 8B depict graphs showing the effect of Compound A and Compound B, administered either alone or in combination, on primarily CLL and iNHL (FL) samples, respectively.

FIG. 9 depicts a graph showing the inhibition of basophil activation by Compound A and Compound B, administered either alone or in combination.

FIGS. 10A and 10B depict graphs showing the effect of administering a Syk inhibitor (Compound C) to WSU-FSCCL parental and resistant cells.

FIGS. 11A and 11B depict two graphs showing the effect of Compound D and/or Compound B on CCL3 and CCL4 secretion from DHL-4 cells.

FIGS. 12A and 12B depict two graphs showing the effect of Compound D and/or Compound B on pMEK and pERK in DHL-4 cells.

### DETAILED DESCRIPTION

The following description sets forth exemplary methods, compositions, kits and articles of manufacture for the treatment of cancer. It should be recognized, however, that such description is not intended as a limitation on the scope of the present disclosure but is instead provided as a description of exemplary embodiments.

Provided are methods for treating a cancer in a subject by administering a combination of small molecule kinase inhibitors. The cancer may be a hematological malignancy, such as leukemia, lymphoma, or multiple myeloma. The subject may be a human. For example, in some embodiments, provided is a method for treating leukemia by administering a combination of small molecule kinase inhibitors that can inhibit B-cell receptor (BCR)-mediated signaling pathways and disrupt essential chronic lymphocytic leukemia (CLL) cell-microenvironment interactions. The methods provided herein may have the effect of inhibiting multiple nodes in the BCR pathway. Simultaneous inhibition of multiple pathways downstream of the BCR may result in a synergistic response that can help with overcoming the resistance observed with single compound use. Thus, dual inhibition may enhance antitumor effects in leukemia, including, for example, chronic lymphocytic leukemia (CLL).

Provided are also compositions (including pharmaceutical compositions, formulations, or unit dosages), articles of manufacture and kits comprising two or more inhibitors selected from a PI3Kδ inhibitor, a Syk inhibitor, and a Btk inhibitor are also provided. For example, the two or more inhibitors are two inhibitors: (i) a PI3Kδ inhibitor, or a pharmaceutically acceptable salt thereof, and (ii) a Syk inhibitor, or a pharmaceutically acceptable salt thereof.

The administration of (S)-2-(1-(9H-purin-6-ylamino)propyl)-5-fluoro-3-phenylquinazolin-4(3H)-one (S-enantiomer of Compound A) or (S)-2-(1-((9H-purin-6-yl)amino)ethyl)-6-fluoro-3-phenylquinazolin-4(3H)-one (S-enantiomer of Compound D), which are each a PI3Kδ inhibitor, and 6-(1H-indazol-6-yl)-N-(4-morpholinophenyl)imidazo[1,2-a]pyrazin-8-amine (Compound B), which is a Syk inhibitor, to cancer cells has led to unexpected synergistic effects compared to the administration of each compound alone. The unexpected synergistic effects include, but are not limited to, for example, decreased cell viability in cancer cells, inhibition or interference with BCR signaling pathways (including MEK and ERK phosphorylation), and/or reduction in chemokine production (e.g., CCL2, CCL3, CLL4 and CLL22 production). Further, the administration of both compounds to cancer cells unexpectedly restored sensitivity or response of such cancer cells that developed resistance to either compound alone; or unexpectedly increased sensitivity or response of such cancer cells that developed resistance to either compound alone.

### Kinase Inhibitors

The small molecule kinase inhibitors used or included in the methods, compositions, kits and articles of manufacture described herein may be selected from a PI3 kinase delta-specific isoform (PI3Kδ) inhibitor, a spleen tyrosine kinase (Syk) inhibitor, and a Bruton's tyrosine kinase (Btk) inhibitor.

In some embodiments, a PI3Kδ inhibitor and a Syk inhibitor is used or included in the methods, compositions, kits and articles of manufacture described herein.

In certain embodiments, the PI3Kδ inhibitor is Compound A, which has the structure:

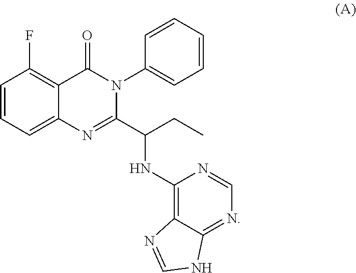 [SMILES: CCC(Nc1ncnc2[nH]cnc12)c1nc2cccc(F)c2c(=O)n1-c1ccccc1]

In one embodiment, Compound A is predominantly the S-enantiomer, having the structure:

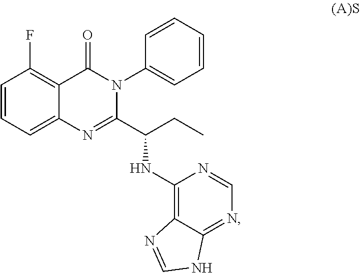 [SMILES: CC[C@H](Nc1ncnc2[nH]cnc12)c1nc2cccc(F)c2c(=O)n1-c1ccccc1]

The (S)-enantiomer of Compound A may also be referred to by its compound name: (S)-2-(1-(9H-purin-6-ylamino)propyl)-5-fluoro-3-phenylquinazolin-4(3H)-one.

In certain embodiments, the PI3Kδ inhibitor is Compound D, which has the structure:

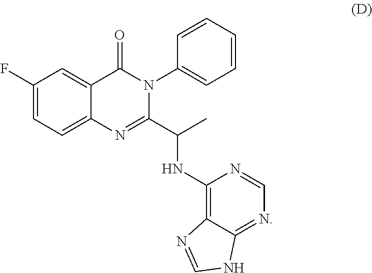 [SMILES: CC(Nc1ncnc2[nH]cnc12)c1nc2ccc(F)cc2c(=O)n1-c1ccccc1]

In one embodiment, Compound D is predominantly the S-enantiomer, having the structure:

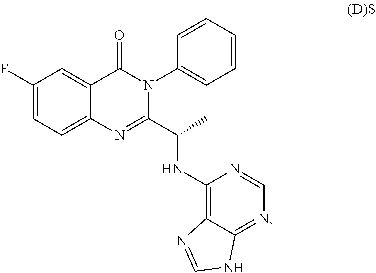 [SMILES: C[C@H](Nc1ncnc2[nH]cnc12)c1nc2ccc(F)cc2c(=O)n1-c1ccccc1]

The compound names provided are named using ChemBioDraw Ultra 12.0 and one skilled in the art understands that the compound structures may be named or identified using other commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS) and International Union of Pure and Applied Chemistry (IUPAC). Accordingly, the compound structure provided above for Compound A may also be named or identified as 5-fluoro-3-phenyl-2-[(1S)-1-(9H-purin-6-ylamino)propyl]quinazolin-4(3H)-one under IUPAC and 5-fluoro-3-phenyl-2-[(1S)-1-(9H-purin-6-ylamino)propyl]-4(3H)-quinazolinone under CAS.

Methods for synthesizing Compounds A and C have been previously described in U.S. Pat. No. 7,932,260. This reference is hereby incorporated herein by reference in its entirety, and specifically with respect to the synthesis of Compounds A and C.

In one embodiment, the Syk inhibitor is Compound B, which has the structure:

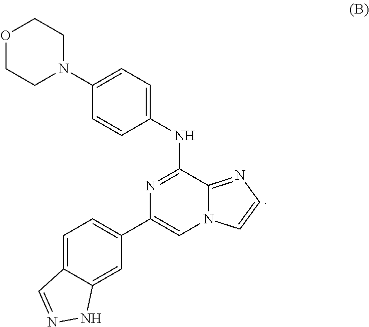 [SMILES: c1cn2cc(-c3ccc4cn[nH]c4c3)nc(Nc3ccc(N4CCOCC4)cc3)c2n1]

Compound B may also be referred to by its compound name: 6-(1H-indazol-6-yl)-N-(4-morpholinophenyl)imidazo[1,2-a]pyrazin-8-amine. The compound name provided is named using ChemBioDraw Ultra 12.0, and one skilled in the art understands that the compound structure may be named or identified using other commonly recognized nomenclature systems and symbols including CAS and IUPAC.

One method for synthesizing Compound B has been previously described in U.S. Pat. No. 8,450,321. This reference is hereby incorporated herein by reference in its entirety, and specifically with respect to the synthesis of Compound B.

In other embodiments, the Syk inhibitor is R406, fostamatinib, BAY-61-3606, NVP-QAB 205 AA, R112, or R343, or a pharmaceutically acceptable salt thereof. See Kaur et al., European Journal of Medicinal Chemistry 67 (2013) 434-446. Therefore, it is understood that in one variation, the methods, compositions, kits and articles of manufacture described herein use or include R406, fostamatinib, BAY-61-3606, NVP-QAB 205 AA, R112, or R343, or a pharmaceutically acceptable salt thereof, as a Syk inhibitor and a PI3Kδ inhibitor such as Compound A or Compound D, or a pharmaceutically acceptable salt thereof.

In some embodiments, the methods, compositions, kits and articles of manufacture described herein use or include Compound A or Compound D, or a pharmaceutically acceptable salt thereof, as the PI3Kδ inhibitor and Compound B, or a pharmaceutically acceptable salt thereof, as a Syk inhibitor. In some embodiments, the methods, compositions, kits and articles of manufacture described herein use or include Compound A or Compound D, or a pharmaceutically acceptable salt thereof, as the PI3Kδ inhibitor and Compound C, or a pharmaceutically acceptable salt thereof, as a Syk inhibitor.

The methods, compositions, kits and articles of manufacture provided may use or include compounds (e.g., Compound A or Compound D, and Compound B), or pharmaceutically acceptable salts, prodrugs, or solvates thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of compounds (e.g., Compound A or Compound D, and Compound B), or pharmaceutically acceptable salts, prodrugs, or solvates thereof, when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

As used herein, by “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, p-toluenesulfonate, 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

A “prodrug” includes any compound that becomes Compound A or Compound D, or Compound B when administered to a subject, e.g., upon metabolic processing of the prodrug.

A “solvate” is formed by the interaction of a solvent and a compound. The compounds used in the methods and compositions (including, for example, pharmaceutical compositions, articles of manufacture and kits) may use or include solvates of salts of Compound A or Compound D and/or Compound B. In one embodiment, the solvent may be hydrates of Compound A or Compound D and/or Compound B.

The methods, compositions, kits and articles of manufacture provided may use or include optical isomers, racemates, or other mixtures thereof, of Compound A or Compound D, or a pharmaceutically acceptable salt, prodrug, or solvate thereof. In those situations, the single enantiomer or diastereomer, i.e., optically active form, can be obtained by asymmetric synthesis or by resolution of the racemate. Resolution of racemates can be accomplished, for example, by conventional methods such as crystallization in the presence of a resolving agent, or chromatography, using, for example a chiral high pressure liquid chromatography (HPLC) column In addition, provided are also Z- and E-forms (or cis- and trans-forms) of Compound A or Compound D, or a pharmaceutically acceptable salt, prodrug, or solvate thereof with carbon-carbon double bonds. The methods, compositions, kits and articles of manufacture provided may use or include any tautomeric form of Compound A or Compound D, or a pharmaceutically acceptable salt, prodrug, or solvate thereof.

In some embodiments, the methods, compositions, kits and articles of manufacture provided may use or include a racemic mixture, or a mixture containing an enantiomeric excess (e.e.) of one enantiomer of Compound A or Compound D. All such isomeric forms of Compound A or Compound D are expressly included herein the same as if each and every isomeric form were specifically and individually listed. In some embodiments, Compound A or Compound D has an enantiomeric excess of at least 60%, at least 65%, at least 70%, at least 75%, at least 80%, at least 85%, at least 90%, at least 95%, at least 98%, or at least 99% of its (S)-enantiomer.

For example, in certain embodiments of the methods, compositions, kits and articles of manufacture provided may use or include: (i) a mixture containing an enantiomeric excess of the (S)-enantiomer of Compound A or Compound D, or a pharmaceutically acceptable salt thereof; and (ii) Compound B, or a pharmaceutically acceptable salt thereof. In other embodiments of the methods, compositions, kits and articles of manufacture provided may use or include: (i) a mixture containing an enantiomeric excess of the (S)-enantiomer of Compound A or Compound D; and (ii) Compound B, or a pharmaceutically acceptable salt thereof.

### Methods for Treating Cancer

Provided herein are methods for treating cancer in a subject (e.g., a human) comprising administering to the subject (e.g., a human) a therapeutically effective amount of a PI3Kδ inhibitor and a therapeutically effective amount of a Syk inhibitor, or pharmaceutically acceptable salts thereof. In some embodiments, the method comprises administering to the subject (e.g., a human) a therapeutically effective amount of Compound A or Compound D, or a pharmaceutically acceptable salt thereof, and a therapeutically effective amount of a Syk inhibitor, or a pharmaceutically acceptable salt thereof. In one embodiment, the method comprises administering to the subject (e.g., a human) a therapeutically effective amount of Compound A or Compound D, or a pharmaceutically acceptable salt thereof, and a therapeutically effective amount of Compound B, or a pharmaceutically acceptable salt thereof. In other embodiment, the method comprises administering to a human in need thereof a therapeutically effective amount of Compound A or Compound D, or a pharmaceutically acceptable salt thereof and a therapeutically effective amount of Compound B, or a pharmaceutically acceptable salt thereof; and the human having or is suspect of having a cancer.

Types of Cancers

The methods described herein may be used to treat various types of cancers. In some embodiments, the cancer may be a hematological malignancy, including relapsed or refractory hematologic malignancies. Cancers amenable to treatment using the methods described herein may include leukemias, lymphomas, and multiple myeloma. Leukemias may include, for example, lymphocytic leukemias and chronic myeloid (myelogenous) leukemias. Lymphomas may include, for example, malignant neoplasms of lymphoid and reticuloendothelial tissues, such as Burkitt's lymphoma, Hodgkin's lymphoma, non-Hodgkin's lymphomas (including, for example, indolent non-Hodgkin's lymphoma), and lymphocytic lymphomas.

In some embodiments, the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL). In one embodiment, the cancer is minimal residual disease (MRD).

In certain embodiments, the cancer is acute lymphocytic leukemia (ALL), B-cell ALL, acute myeloid leukemia (AML), chronic lymphocytic leukemia (CLL), small lymphocytic lymphoma (SLL), multiple myeloma (MM), non-Hodgkin's lymphoma (NHL), indolent NHL (iNHL), mantle cell lymphoma (MCL), follicular lymphoma, Waldenstrom's macroglobulinemia (WM), B-cell lymphoma, or diffuse large B-cell lymphoma (DLBCL).

In some embodiments, provided are methods of treating cancer in a subject (e.g., a human) by administering to the subject a therapeutically effective amount of Compound A or Compound D, or a pharmaceutically acceptable salt thereof, and a therapeutically effective amount of Compound B, or a pharmaceutically acceptable salt thereof, wherein the cancer is leukemia. In some embodiments, the leukemia is chronic leukemia. An example of chronic leukemia is chronic lymphocytic leukemia (CLL). In one embodiment, the leukemia is minimal residual disease (MRD).

In other embodiments, provided are also methods of treating cancer in a subject by administering to the subject (e.g. a human) a therapeutically effective amount of Compound A or Compound D, or a pharmaceutically acceptable salt thereof, and a therapeutically effective amount of Compound B, or a pharmaceutically acceptable salt thereof, wherein the cancer is lymphoma. In some embodiments, the lymphoma is non-Hodgkin's lymphoma (NHL). An example of non-Hodgkin's lymphoma is indolent NHL (iNHL), or refractory iNHL.

Any of the methods of treatment provided may be used to treat cancer at an advanced stage. Any of the methods of treatment provided herein may be used to treat cancer at locally advanced stage. Any of the methods of treatment provided herein may be used to treat early stage cancer. Any of the methods of treatment provided herein may be used to treat cancer in remission. In some of the embodiments of any of the methods of treatment provided herein, the cancer has reoccurred after remission. In some embodiments of any of the methods of treatment provided herein, the cancer is progressive cancer.

Subjects

Any of the methods of treatment provided may be used to treat a subject (e.g., human) who has been diagnosed with or is suspected of having cancer. As used herein, a “subject” includes a mammal, including, for example, a human.

In some embodiments, the subject may be a human who exhibits one or more symptoms associated with cancer. In some embodiments, the subject is at an early stage of a cancer. In other embodiments, the subject is at an advanced stage of cancer.

In some of the embodiments of any of the methods of treatment provided herein, the subject may be a human who is at risk, or genetically or otherwise predisposed (e.g., risk factor) to developing cancer who has or has not been diagnosed with cancer. As used herein, an “at risk” subject is a subject who is at risk of developing cancer. The subject may or may not have detectable disease, and may or may not have displayed detectable disease prior to the treatment methods described herein. An at risk subject may have one or more so-called risk factors, which are measurable parameters that correlate with development of cancer, which are described herein. A subject having one or more of these risk factors has a higher probability of developing cancer than an individual without these risk factor(s).

In some embodiments, these risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced this disease, and those whose risk is determined by analysis of genetic or biochemical markers.

In some embodiments, the subject may be a human who is undergoing one or more standard therapies for treating cancer, such as chemotherapy, radiotherapy, immunotherapy, and/or surgery. Thus, in some foregoing embodiments, Compound A and Compound B, or Compound D and Compound B may be administered before, during, or after administration of chemotherapy, radiotherapy, immunotherapy, and/or surgery.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In certain embodiments, the subject is refractory to at least one, at least two, at least three, or at least four chemotherapy treatment (including standard or experimental chemotherapy) selected from fludarabine, rituximab, obinutuzumab, alkylating agents, alemtuzumab and other chemotherapy treatments such as CHOP (cyclophosphamide, doxorubicin, vincristine, prednisone); R-CHOP (rituximab-CHOP); hyperCVAD (hyperfractionated cyclophosphamide, vincristine, doxorubicin, dexamethasone, methotrexate, cytarabine); R-hyperCVAD (rituximab-hyperCVAD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (dexamethasone, thalidomide, cisplatin, Adriamycin®, cyclophosphamide, etoposide).

Other examples of chemotherapy treatments (including standard or experimental chemotherapies) are described below. In addition, treatment of certain lymphomas is reviewed in Cheson, B. D., Leonard, J. P., “Monoclonal Antibody Therapy for B-Cell Non-Hodgkin's Lymphoma” The New England Journal of Medicine 2008, 359(6), p. 613-626; and Wierda, W. G., “Current and Investigational Therapies for Patients with CLL” Hematology 2006, p. 285-294. Lymphoma incidence patterns in the United States is profiled in Morton, L. M., et al. “Lymphoma Incidence Patterns by WHO Subtype in the United States, 1992-2001” Blood 2006, 107(1), p. 265-276.

For example, treatment of non-Hodgkin's lymphomas (NHL), especially of B cell origin, include the use of monoclonal antibodies, standard chemotherapy approaches (e.g., CHOP, CVP, FCM, MCP, and the like), radioimmunotherapy, and combinations thereof, especially integration of an antibody therapy with chemotherapy. Examples of unconjugated monoclonal antibodies for Non-Hodgkin's lymphoma/B-cell cancers include rituximab, alemtuzumab, human or humanized anti-CD20 antibodies, lumiliximab, anti-TRAIL, bevacizumab, galiximab, epratuzumab, SGN-40, and anti-CD74. Examples of experimental antibody agents used in treatment of Non-Hodgkin's lymphoma/B-cell cancers include ofatumumab, ha20, PRO131921, alemtuzumab, galiximab, SGN-40, CHIR-12.12, epratuzumab, lumiliximab, apolizumab, milatuzumab, and bevacizumab. Examples of standard regimens of chemotherapy for Non-Hodgkin's lymphoma/B-cell cancers include CHOP (cyclophosphamide, doxorubicin, vincristine, prednisone), FCM (fludarabine, cyclophosphamide, mitoxantrone), CVP (cyclophosphamide, vincristine and prednisone), MCP (mitoxantrone, chlorambucil, and prednisolone), R-CHOP (rituximab plus CHOP), R-FCM (rituximab plus FCM), R-CVP (rituximab plus CVP), and R-MCP (R-MCP). Examples of radioimmunotherapy for Non-Hodgkin's lymphoma/B-cell cancers include yttrium-90-labeled ibritumomab tiuxetan, and iodine-131-labeled tositumomab.

In another example, therapeutic treatments for mantle cell lymphoma (MCL) include combination chemotherapies such as CHOP (cyclophosphamide, doxorubicin, vincristine, prednisone), hyperCVAD (hyperfractionated cyclophosphamide, vincristine, doxorubicin, dexamethasone, methotrexate, cytarabine) and FCM (fludarabine, cyclophosphamide, mitoxantrone). In addition, these regimens can be supplemented with the monoclonal antibody rituximab (Rituxan) to form combination therapies R-CHOP, hyperCVAD-R, and R-FCM. Other approaches include combining any of the abovementioned therapies with stem cell transplantation or treatment with ICE (iphosphamide, carboplatin and etoposide). Other approaches to treating mantle cell lymphoma includes immunotherapy such as using monoclonal antibodies like Rituximab (Rituxan). Rituximab can be used for treating indolent B-cell cancers, including marginal-zone lymphoma, WM, CLL and small lymphocytic lymphoma. A combination of Rituximab and chemotherapy agents is especially effective. A modified approach is radioimmunotherapy, wherein a monoclonal antibody is combined with a radioisotope particle, such as Iodine-131 tositumomab (Bexxar®) and Yttrium-90 ibritumomab tiuxetan (Zevalin®). In another example, Bexxar® is used in sequential treatment with CHOP. Another immunotherapy example includes using cancer vaccines, which is based upon the genetic makeup of an individual patient's tumor. A lymphoma vaccine example is GTOP-99 (MyVax®). Yet other approaches to treating mantle cell lymphoma includes autologous stem cell transplantation coupled with high-dose chemotherapy, or treating mantle cell lymphoma includes administering proteasome inhibitors, such as Velcade® (bortezomib or PS-341), or antiangiogenesis agents, such as thalidomide, especially in combination with Rituxan. Another treatment approach is administering drugs that lead to the degradation of Bcl-2 protein and increase cancer cell sensitivity to chemotherapy, such as oblimersen (Genasense) in combination with other chemotherapeutic agents. Another treatment approach includes administering mTOR inhibitors, which can lead to inhibition of cell growth and even cell death; a non-limiting example is Temsirolimus (CCI-779), and Temsirolimus in combination with Rituxan®, Velcade® or other chemotherapeutic agents.

Other recent therapies for MCL have been disclosed (Nature Reviews; Jares, P. 2007). Such examples include Flavopiridol, PD0332991, R-roscovitine (Selicilib, CYC202), Styryl sulphones, Obatoclax (GX15-070), TRAIL, Anti-TRAIL DR4 and DR5 antibodies, Temsirolimus (CCl-779), Everolimus (RAD001), BMS-345541, Curcumin, Vorinostat (SAHA), Thalidomide, lenalidomide (Revlimid®, CC-5013), and Geldanamycin (17-AAG).

Examples of other therapeutic agents used to treat Waldenstrom's Macroglobulinemia (WM) include perifosine, bortezomib (Velcade®), rituximab, sildenafil citrate (Viagra®), CC-5103, thalidomide, epratuzumab (hLL2-anti-CD22 humanized antibody), simvastatin, enzastaurin, campath-1H, dexamethasone, DT PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine I-131 tositumomab, motexafin gadolinium, arsenic trioxide, tipifarnib, autologous human tumor-derived HSPPC-96, veltuzumab, bryostatin 1, and PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

Examples of other therapeutic agents used to treat diffuse large B-cell lymphoma (DLBCL) drug therapies (Blood 2005 Abramson, J.) include cyclophosphamide, doxorubicin, vincristine, prednisone, anti-CD20 monoclonal antibodies, etoposide, bleomycin, many of the agents listed for Waldenstrom's, and any combination thereof, such as ICE and R-ICE.

Examples of other therapeutic agents used to treat chronic lymphocytic leukemia (CLL) (Spectrum, 2006, Fernandes, D.) include Chlorambucil (Leukeran), Cyclophosphamide (Cyloxan, Endoxan, Endoxana, Cyclostin), Fludarabine (Fludara), Pentstatin (Nipent), Cladribine (Leustarin), Doxorubicin (Adriamycin®, Adriblastine), Vincristine (Oncovin), Prednisone, Prednisolone, Alemtuzumab (Campath, MabCampath), many of the agents listed for Waldenstrom's, and combination chemotherapy and chemoimmunotherapy, including the common combination regimen: CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); and FR (fludarabine, rituximab).

Thus, provided is a method of sensitizing a subject who is (i) substantially refractory to at least one chemotherapy treatment, or (ii) is in relapse after treatment with chemotherapy, or both (i) and (ii), wherein the method comprises administering to the subject an effective amount of Compound A, and an effective amount of Compound B or a pharmaceutically acceptable salt thereof. A subject who is sensitized is a subject who is responsive to the treatment involving administration of Compound A and Compound B, or who has not developed resistance to such treatment.

The treatment involving administration of Compound A and Compound B, can also sensitize, or restore sensitivity of, cells that may otherwise be resistant, have developed resistance, or not responsive, to killing or apoptosis by chemotherapy treatments or by administration of a PI3K-δ inhibitor (such as Compound A or Compound D) alone. Cancer cells that are sensitized, or have restored sensitivity, are cancer cells that are responsive to the treatment involving administration of Compound A and Compound B, or Compound D and Compound B. In some embodiments, the administration of both compounds sensitizes, or restores sensitivity of, such cancer cells by increasing the level of reduction in cell viability. In certain embodiments, the administration of Compound A and Compound B, or Compound D and Compound B increases the level of reduction in cell viability by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to contact with only Compound A or Compound D or contact with only Compound B. In other embodiments, the administration of Compound A and Compound B, or Compound D and Compound B increases the level of reduction in cell viability by between 10% and 99%, between 10% and 90%, between 10% and 80%, between 10% and 70%, between 20% and 99%, between 20% and 90%, between 20% and 80%, between 25% and 95%, between 25% and 90%, between 25% and 80%, between 25% and 75%, or between 30% and 90%.

Treatment

As used herein, “treatment” or “treating” is an approach for obtaining beneficial or desired results including clinical results. For example, beneficial or desired clinical results may include one or more of the following:

(i) decreasing one more symptoms resulting from the disease;

(ii) diminishing the extent of the disease, stabilizing the disease (e.g., preventing or delaying the worsening of the disease);

(iii) preventing or delaying the spread (e.g., metastasis) of the disease;

(iv) preventing or delaying the occurrence or recurrence of the disease, delay or slowing the progression of the disease;

(v) ameliorating the disease state, providing a remission (whether partial or total) of the disease, decreasing the dose of one or more other medications required to treat the disease;

(vi) delaying the progression of the disease, increasing the quality of life, and/or

(vii) prolonging survival.

In one variation, the administration of Compound A and Compound B, or Compound D and Compound B or pharmaceutically acceptable salts thereof, decreases the severity of the cancer. In one aspect, the decrease in the severity of the cancer may be assessed by chemokine levels (e.g., CCL2, CCL3, CCL4, CCL22) by the methods described herein.

In some embodiments, the administration of Compound A and Compound B, or Compound D and Compound B or pharmaceutically acceptable salts thereof, may reduce the severity of one or more symptoms associated with cancer by at least about any of 10%, 20%, 30%, 40%, 50%, 60%, 70%, 80%, 90%, 95% or 100% compared to the corresponding one or more symptoms in the same subject prior to treatment or compared to the corresponding symptom in other subjects not receiving the composition. In certain embodiments, treatment or treating may also include a reduction of pathological consequence of cancer. The methods provided contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or subject being treated. As is evident to one of skill in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams, mammography, imaging, or biopsy. Development may also refer to disease progression that may be initially undetectable and includes occurrence, recurrence, and onset.

In certain embodiments, the methods may be used to treat the growth or proliferation of cancer cells of hematopoietic origin. For example, the cancer cells may be of lymphoid origin. In one embodiment, the cancer cells are related to or derived from B lymphocytes or B lymphocyte progenitors.

The administration of both Compound A and Compound B, or both Compound D and Compound B or pharmaceutically acceptable salts thereof, may decrease cell viability of cancer cells, disrupt or inhibit phosphorylation in certain metabolic pathways, and/or reduce or inhibit certain chemokine production that may correlate with reducing disease severity.

Thus, provided herein are also methods for decreasing cell viability in cancer cells in a human, comprising administering to the human Compound A and Compound B, or Compound D and Compound B or pharmaceutically acceptable salts thereof, in amounts sufficient to detectably decrease cell viability in the cancer cells. Provided herein are also methods for decreasing cell viability in cancer cells, comprising administering to the human or contacting the cancer cells with Compound A and Compound B, or Compound D and Compound B or pharmaceutically acceptable salts thereof, in amounts sufficient to detectably decrease cell viability in the cancer cells. In some embodiments, the cell viability in the cancer cells after administering to the human, or contacting the cancer cells with, Compound A and Compound B, or Compound D and Compound B or pharmaceutically acceptable salts thereof, is decreased by at least 10%, at least 20%, at least 30%, at least 40%, at least 50%, at least 60%, at least 70%, at least 80%, or at least 90% compared to cell viability in cancer cells in the absence of Compound A and Compound B, or Compound D and Compound B or pharmaceutically acceptable salts thereof. In certain embodiments, the cell viability in cancer cells after administering to the human, or contacting the cancer cells with, Compound A and Compound B, or Compound D and Compound B or pharmaceutically acceptable salts thereof, is decreased by between 10% and 99%, between 10% and 90%, between 10% and 80%, between 20% and 90%, between 20% and 80%, between 20% and 70% compared to cell viability in cancer cells in the absence of Compound A and Compound B, or Compound D and Compound B or pharmaceutically acceptable salts thereof. In one embodiment of the foregoing methods, the cancer cells are chronic lymphocytic leukemia (CLL) cells.

Any suitable methods, techniques and assays known in the art may be used to measure cell viability. For example, in one embodiment, cell viability in cancer cells, such as CLL cells, may be determined by a cell viability assay, such as MTS assay. Other suitable assays may include, for example, the use of suitable stains, dyes, polynucleotide, polypeptide, or biomarkers.

Provided herein are also methods for decreasing AKT phosphorylation, S6 phosphorylation, or AKT and S6 phosphorylation in cancer cells in a human, comprising administering to the human Compound A and Compound B, or pharmaceutically acceptable salts thereof, in amounts sufficient to detectably decrease AKT phosphorylation, S6 phosphorylation, or AKT and S6 phosphorylation in the cancer cells. Provided herein are also methods for decreasing AKT phosphorylation, S6 phosphorylation, or AKT and S6 phosphorylation in cancer cells, comprising administering to the human or contacting cancer cells with Compound A and Compound B, or pharmaceutically acceptable salts thereof, in amounts sufficient to detectably decrease AKT phosphorylation, S6 phosphorylation, or AKT and S6 phosphorylation in the cancer cells. In some embodiments, S6 phosphorylation in the cancer cells after administering to the human, or contacting the cancer cells with, Compound A and Compound B, or pharmaceutically acceptable salts thereof, is decreased by at least 10%, at least 20%, at least 30%, at least 40%, at least 50%, at least 60%, at least 70%, at least 80%, or at least 90% compared to S6 phosphorylation in cancer cells in the absence of Compound A and Compound B, or the absence of Compound D and Compound B or pharmaceutically acceptable salts thereof. In certain embodiments, S6 phosphorylation in cancer cells after administering to the human, or contacting the cancer cells with, Compound A and Compound B, or Compound D and Compound B or pharmaceutically acceptable salts thereof, is decreased by between 10% and 99%, between 10% and 90%, between 10% and 80%, between 20% and 90%, between 20% and 80%, between 20% and 70% compared to S6 phosphorylation in cancer cells in the absence of Compound A and Compound B, or the absence of Compound D and Compound B or pharmaceutically acceptable salts thereof. In one embodiment of the foregoing methods, the cancer cells are chronic lymphocytic leukemia (CLL) cells.

Provided herein are also methods for decreasing AKT phosphorylation, ERK phosphorylation, or AKT and ERK phosphorylation in cancer cells in a human, comprising administering to a human Compound A and Compound B, or Compound D and Compound B or pharmaceutically acceptable salts thereof, in amounts sufficient to detectably decrease AKT phosphorylation, ERK phosphorylation, or AKT and ERK phosphorylation in the cancer cells. Provided herein are also methods for decreasing AKT phosphorylation, ERK phosphorylation, or AKT and ERK phosphorylation in cancer cells, comprising contacting cancer cells with Compound A and Compound B, or Compound D and Compound B or pharmaceutically acceptable salts thereof, in amounts sufficient to detectably decrease AKT phosphorylation, ERK phosphorylation, or AKT and ERK phosphorylation in the cancer cells. In some embodiments, ERK phosphorylation in the cancer cells after administering to the human or contacting the cancer cells with, Compound A and Compound B, or Compound D and Compound B or pharmaceutically acceptable salts thereof, is decreased by at least 10%, at least 20%, at least 30%, at least 40%, at least 50%, at least 60%, at least 70%, at least 80%, or at least 90% compared to ERK phosphorylation in cancer cells in the absence of Compound A and Compound B, or the absence of Compound D and Compound B or pharmaceutically acceptable salts thereof. In certain embodiments, ERK phosphorylation in cancer cells after administering to the human, or contacting the cancer cells with, Compound A and Compound B, or Compound D and Compound B or pharmaceutically acceptable salts thereof, is decreased by between 10% and 99%, between 10% and 90%, between 10% and 80%, between 20% and 90%, between 20% and 80%, between 20% and 70% compared to ERK phosphorylation in cancer cells in the absence of Compound A and Compound B, or the absence of Compound D and Compound B or pharmaceutically acceptable salts thereof. In one embodiment of the foregoing methods, the cancer cells are Burkitt's lymphoma cells.

Any suitable methods, techniques and assays known in the art may be used to measure AKT phosphorylation, S6 phosphorylation, and ERK phosphorylation. For example, in one embodiment, AKT phosphorylation, S6 phosphorylation, and/or ERK phosphorylation in cancer cells, such as CLL cells or Burkitt's lymphoma cells, may be determined by flow cytometry or immunoblotting.

Provided herein also are methods for decreasing chemokine production in a sample comprising cells expressing CCL2, CCL3, CCL4, CCL22, or any combinations thereof, comprising contacting the sample with Compound A and Compound B, or pharmaceutically acceptable salts thereof, in amounts sufficient to detectably chemokine production in the sample.

In some embodiments, one or more of the following (i)-(iv) applies:

(i) CLL2 production after contact with Compound A and Compound B, or Compound D and Compound B or pharmaceutically acceptable salts thereof, is decreased by at least 5%, at least 10%, at least 20%, at least 30%, at least 40%, at least 50%, at least 60%, at least 70%, at least 80%, or at least 90% compared to CLL2 production in the cells in the absence of Compound A and Compound B, or the absence of Compound D and Compound B or pharmaceutically acceptable salts thereof;

(ii) CLL3 production after contact with Compound A and Compound B, or Compound D and Compound B or pharmaceutically acceptable salts thereof, is decreased by at least 5%, at least 10%, at least 20%, at least 30%, at least 40%, at least 50%, at least 60%, at least 70%, at least 80%, or at least 90% compared to CLL3 production in the cells in the absence of Compound A and Compound B, or the absence of Compound D and Compound B or pharmaceutically acceptable salts thereof;

(iii) CLL4 production after contact with Compound A and Compound B, or Compound D and Compound B or pharmaceutically acceptable salts thereof, is decreased by at least 5%, at least 10%, at least 20%, at least 30%, at least 40%, at least 50%, at least 60%, at least 70%, at least 80%, or at least 90% compared to CLL4 production in the cells in the absence of Compound A and Compound B, or the absence of Compound D and Compound B or pharmaceutically acceptable salts thereof; and

(iv) CLL22 production after contact with Compound A and Compound B, or Compound D and Compound B or pharmaceutically acceptable salts thereof, is decreased by at least 5%, at least 10%, at least 20%, at least 30%, at least 40%, at least 50%, at least 60%, at least 70%, at least 80%, or at least 90% compared to CLL22 production in the cells in the absence of Compound A and Compound B, or the absence of Compound D and Compound B or pharmaceutically acceptable salts thereof.

It is intended and understood that each and every variation of the decrease in production of any one of the chemokines provided above may be combined with each and every variation of the other chemokines, as if each and every combination is individually described. For example, CCL3, CCL4, CXCL12, CXCL13, tumor necrosis factor alpha, and c-creative protein may be suitable chemokines.

Any suitable methods, techniques and assays known in the art may be used to determine the levels of the chemokines in a sample. For example, immunoassays (or immunological binding assays) may be used to qualitatively or quantitatively analyze the chemokine levels in a sample. A general overview of the applicable technology can be found in a number of readily available manuals, e.g., Harlow & Lane, Cold Spring Harbor Laboratory Press, Using Antibodies: A Laboratory Manual (1999) Immunoassays typically use an antibody that specifically binds to a protein or antigen of choice. The antibody may be produced by any of a number of means well known to those of skill in the art.

### Dosing Regimen, Order of Administration, and Route of Administration

Compound A and Compound B, or pharmaceutically acceptable salts thereof, and pharmaceutical compositions thereof, may include those wherein the compounds are administered in a therapeutically effective amount to achieve its intended purpose. As used herein, a “therapeutically effective amount” means an amount sufficient to modulate PI3Kδ and/or Syk expression or activity, and thereby treat a subject (such as a human) suffering an indication, or to alleviate the existing symptoms of the indication. Determination of a therapeutically effective amount is well within the capability of those skilled in the art, especially in light of the detailed disclosure provided herein. In some embodiments, a therapeutically effective amount of Compound A or Compound D and a therapeutically effective amount of Compound B may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

The dosing regimen of Compound A and Compound B, or Compound D and Compound B or pharmaceutically acceptable salts thereof, in the methods provided herein may vary depending upon the indication, route of administration, and severity of the condition, for example. Depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The formulation and route of administration chosen may be tailored to the individual subject, the nature of the condition to be treated in the subject, and generally, the judgment of the attending practitioner. For example, the therapeutic index of Compound A and Compound B, or Compound D and Compound B may be enhanced by modifying or derivatizing the compound for targeted delivery to cancer cells expressing a marker that identifies the cells as such. For example, the compounds can be linked to an antibody that recognizes a marker that is selective or specific for cancer cells, so that the compounds are brought into the vicinity of the cells to exert their effects locally, as previously described. See e.g., Pietersz et al., Immunol. Rev., 129:57 (1992); Trail et al., Science, 261:212 (1993); and Rowlinson-Busza et al., Curr. Opin. Oncol., 4:1142 (1992).

Dosing Regimen

The therapeutically effective amount of Compound A or Compound B, or Compound D and Compound B or pharmaceutically acceptable salts thereof, may be provided in a single dose or multiple doses to achieve the desired treatment endpoint. As used herein, “dose” refers to the total amount of an active ingredient (e.g., Compound A or Compound D, Compound B, or pharmaceutically acceptable salts thereof) to be taken each time by a subject (e.g., a human).

Exemplary doses of Compound A or Compound D, or a pharmaceutically acceptable salt thereof, for a human subject may be between about 0.01 mg to about 1500 mg or between about 50 mg to about 200 mg, or about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg. It should be understood that reference to “about” a value or parameter herein includes (and describes) embodiments that are directed to that value or parameter per se. For example, description referring to “about x” includes description of “x” per se.

Exemplary doses of Compound B, or a pharmaceutically acceptable salt thereof, for a human subject may be between about 0.01 mg to about 1800 mg, or between about 0.01 mg to about 1500 mg, or between about 10 mg to about 1500 mg, or between about 10 mg to about 1300 mg, or between about 10 mg to about 1000 mg, or between about 10 mg to about 800 mg, or between about 10 mg to about 600 mg, or between about 10 mg to about 300 mg, or between about 10 mg to about 200 mg, or between about 10 mg to about 100 mg, or between about 100 mg to about 800 mg, or between about 100 mg to about 600 mg, or between about 100 mg to about 300 mg, or between about 100 mg to about 200 mg, or between about 200 mg to about 350 mg, or between about 250 mg to about 300 mg, or between about 200 mg to about 400 mg, or between about 400 mg to about 600 mg, or between about 400 mg to about 800 mg, or between about 600 mg or about 800 mg, or between about 800 mg to about 1200 mg, or between about 1200 mg to about 1600, or between about 50 mg to about 200 mg, or about 25 mg, about 50 mg, about 75 mg, about 100 mg, about 125 mg, or about 150 mg, or about 175 mg, about 200 mg, about 250 mg, about 300 mg, about 350 mg, about 400 mg, about 450 mg, about 500 mg, about 550 mg, about 600 mg, about 650 mg, about 700 mg, or about 750 mg, or about 800 mg, or about 850 mg, or about 900 mg, or about 950 mg, or about 1000 mg, or about 1100 mg, or about 1200 mg, or about 1300 mg, or about 1400 mg, or about 1500 mg, or about 1600 mg, or about 1800 mg.

It is intended and understood that each and every variation of the doses of Compound A or Compound D may be combined with each and every variation of the doses of Compound B, as if each and every combination is individually described. For example, in one embodiment, a 100 mg dose of Compound A or Compound D may be administered with a 200 mg dose of Compound B. In another embodiment, a 100 mg dose of Compound A or Compound D may be administered with a 600 mg dose of Compound B. In yet another embodiment, a 150 mg dose of Compound A or Compound D may be administered with a 600 mg dose of Compound B. In other embodiment, a 150 mg dose of Compound A or Compound D may be administered with a 900 mg dose of Compound B. In yet other embodiment, a 150 mg dose of Compound A or Compound D may be administered with a 900 mg dose of Compound B. In some other embodiment, a 300 mg dose of Compound A or Compound D may be administered with a 200 mg dose of Compound B. In some other embodiment, a 300 mg dose of Compound A or Compound D may be administered with a 900 mg dose of Compound B. In certain embodiment, a 100 mg dose of Compound A or Compound D may be administered with a 900 mg dose of Compound B.

In other embodiments, the methods provided comprise continuing to treat the subject (e.g., a human) by administering the doses of Compound A and Compound B, or Compound D and Compound B or pharmaceutically acceptable salts thereof, at which clinical efficacy is achieved or reducing the doses by increments to a level at which efficacy can be maintained. In a particular embodiment, the methods provided comprise administering to the subject (e.g., a human) an initial daily dose of 20 mg to 500 mg of Compound A or Compound D, or a pharmaceutically acceptable salt thereof, and increasing said dose to a total dosage of 50 mg to 400 mg per day over at least 6 days. Optionally, the dosage can be further increased to about 750 mg per day. In another embodiment, the methods provided comprise administering to the subject (e.g., a human) an initial daily dose of 100 mg to 1000 mg of Compound B, or a pharmaceutically acceptable salt thereof, and increasing said dose to a total dosage of 50 mg to 400 mg per day over at least 6 days. Thus, it should also be understood that the dose(s) of Compound A and/or Compound B, or Compound D and/or Compound B, or pharmaceutically acceptable salts thereof, may be increased by increments until clinical efficacy is achieved. Increments of about 25 mg, about 50 mg, about 100 mg, or about 125 mg, or about 150 mg, or about 200 mg, or about 250 mg, or about 300 mg can be used to increase the dose. The dose can be increased daily, every other day, two, three, four, five or six times per week, or once per week.

The frequency of dosing will depend on the pharmacokinetic parameters of the compounds administered and the route of administration. The dosing frequency for Compound A or Compound D may be the same or different from the dosing frequency for Compound B. In some embodiments, Compound A or Compound D or a pharmaceutically acceptable salt thereof is administered once a day or twice a day. In some embodiments, Compound B or a pharmaceutically acceptable salt thereof is administered once a day or twice a day. In one embodiment, both Compound A and Compound B, or both Compound D and Compound B or pharmaceutically acceptable salts thereof, are both independently administered twice a day. In other embodiment, Compound B or a pharmaceutical acceptable salt thereof and Compound A, are both independently administered twice a day. In other embodiment, Compound B or a pharmaceutical acceptable salt thereof and Compound D, are both independently administered twice a day.

The dose and frequency of dosing may also depend on pharmacokinetic and pharmacodynamic, as well as toxicity and therapeutic efficiency data. For example, pharmacokinetic and pharmacodynamic information about Compound A and Compound B, or Compound D and Compound B, and the formulation of Compound A and Compound B, or Compound D and Compound B can be collected through preclinical in vitro and in vivo studies, later confirmed in humans during the course of clinical trials. Thus, for Compound A and Compound B used in the methods provided, or for Compound D and Compound B used in the methods provided, a therapeutically effective dose can be estimated initially from biochemical and/or cell-based assays. Then, dosage can be formulated in animal models to achieve a desirable circulating concentration range that modulates PI3Kδ and/or Syk expression or activity. As human studies are conducted further information will emerge regarding the appropriate dosage levels and duration of treatment for various diseases and conditions.

Toxicity and therapeutic efficacy of Compound A and Compound B, and Compound D and Compound B can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

The administration of Compound A and Compound B, or Compound D and Compound B or pharmaceutically acceptable salts thereof, may be administered under fed conditions. The term fed conditions or variations thereof refers to the consumption or uptake of food, in either solid or liquid forms, or calories, in any suitable form, before or at the same time when the compounds or pharmaceutical compositions thereof are administered. For example, Compound A and Compound B, or Compound D and Compound B or pharmaceutically acceptable salts thereof, may be administered to the subject (e.g., a human) within minutes or hours of consuming calories (e.g., a meal). In some embodiments, Compound A and Compound B, or Compound D and Compound B or pharmaceutically acceptable salts thereof, may be administered to the subject (e.g., a human) within 5-10 minutes, about 30 minutes, or about 60 minutes consuming calories.

Order of Administration

The order of administering Compound A and Compound B, or Compound D and Compound B or pharmaceutically acceptable salts thereof, may also vary. In some embodiments, Compound A and Compound B, or Compound D and Compound B or pharmaceutically acceptable salts thereof, may be administered sequentially (e.g., sequential administration) or simultaneously (e.g., simultaneous administration). For example, in some embodiments, Compound A or Compound D or a pharmaceutically acceptable salt thereof is administered before Compound B or a pharmaceutically acceptable salt thereof. In other embodiments, Compound B or a pharmaceutically acceptable salt thereof is administered before Compound A or Compound D or a pharmaceutically acceptable salt thereof. In yet other embodiments, Compound A or Compound D or a pharmaceutically acceptable salt thereof, and Compound B or a pharmaceutically acceptable salt thereof, are administered simultaneously.

In some embodiments, Compound A and Compound B, or Compound D and Compound B or pharmaceutically acceptable salts thereof, may be administered sequentially. In certain embodiments, sequential administration means that Compound A and Compound B, or Compound D and Compound B or pharmaceutically acceptable salts thereof, are administered with a time separation of several minutes, hours, days, or weeks. In certain embodiments, the compounds are administered with a time separation of at least 15 minutes, at least 30 minutes, at least 60 minutes, or 1 day, 2 days, 3 days, 4 days, 5 days, 6 days, or 7 days, or 2 weeks, 3 weeks, 4 weeks, 5 weeks, 6 weeks, 7 weeks, or 8 weeks. In one embodiment, Compound A or Compound D, or a pharmaceutically acceptable salt thereof, may be administered before Compound B, or a pharmaceutically acceptable salt thereof. In another embodiment, Compound B, or a pharmaceutically acceptable salt thereof, may be administered before Compound A or Compound D, or a pharmaceutically acceptable salt thereof. When administered sequentially, Compound A and Compound B, or Compound D and Compound B or pharmaceutically acceptable salts thereof, may be administered in two or more administrations. When administered sequentially, Compound A and Compound B, or Compound D and Compound B or pharmaceutically acceptable salts thereof, are contained in separate compositions, which may be contained in the same or different packages.

In other embodiments, Compound A and Compound B, or Compound D and Compound B or pharmaceutically acceptable salts thereof, may be administered simultaneously. In certain embodiments, simultaneous administration means that Compound A and Compound B, or Compound D and Compound B or pharmaceutically acceptable salts thereof, the compounds are administered with a time separation of no more than a few minutes or seconds. In certain embodiments, the compounds are administered with a time separate of no more than about 15 minutes, about 10 minutes, about 5 minutes, or 1 minute. When administered simultaneously, in certain embodiments, Compound A and Compound B, or Compound D and Compound B or pharmaceutically acceptable salts thereof, are contained in separate compositions, which may be contained in the same or different packages. In other embodiments, Compound A and Compound B, or Compound D and Compound B or pharmaceutically acceptable salts thereof, are contained in the same composition.

Further, in some embodiments, the administration of Compound A and Compound B, or pharmaceutically acceptable salts thereof can be combined with supplemental doses of either or both Compound A and Compound B, or pharmaceutically acceptable salts thereof. In other embodiments, the administration of Compound D and Compound B, or pharmaceutically acceptable salts thereof can be combined with supplemental doses of either or both Compound D and Compound B, or pharmaceutically acceptable salts thereof.

The administration of Compound A and Compound B, or Compound D and Compound B or pharmaceutically acceptable salts thereof, may provide unexpected “synergy” or “synergistic effect(s)”, i.e., the effect achieved when the active ingredients used together is greater than the sum of the effects that results from using the compounds separately or greater than the additive effects resulted from the compound alone. A synergistic effect may be attained when the active ingredients (e.g., Compound A and Compound B, or Compound D and Compound B or pharmaceutically acceptable salts thereof) are: (1) co-formulated and administered or delivered simultaneously in a combined formulation; (2) delivered sequentially or simultaneously as separate formulations; or (3) by some other regimen. In certain embodiments, a synergistic effect may be attained when the compounds are administered or delivered sequentially, e.g., in separate tablets, pills or capsules, or by different injections in separate syringes.

Compound A and Compound B, or pharmaceutically acceptable salts thereof may be administered in the same or separate compositions. Similarly, Compound D and Compound B, or pharmaceutically acceptable salts thereof may be administered in the same or separate compositions. For example, in some embodiments, the active ingredients may be administered separately in a pharmaceutical composition comprising Compound A or Compound D and a pharmaceutical composition comprising Compound B or a pharmaceutically acceptable salt thereof.

Modes of Administration

Compound A and Compound B, or Compound D and Compound B or pharmaceutically acceptable salts thereof, may be administered by any conventional method, including parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral, buccal, sublingual, and rectal administration. Transepithelial administration modalities include, for example, transmucosal administration and transdermal administration. Transmucosal administration includes, for example, enteral administration as well as nasal, inhalation, and deep lung administration; vaginal administration; and buccal and sublingual administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., POWDERJECT™.

In some embodiments, Compound A and Compound B, or Compound D and Compound B or pharmaceutically acceptable salts thereof, may be independently administered orally, intravenously or by inhalation. In one embodiment, Compound B or pharmaceutically acceptable salts thereof and Compound A or Compound D, are both administered orally.

In one embodiment, Compound A or Compound D, or a pharmaceutically acceptable salt thereof, is administered orally. In some embodiments, Compound A or Compound D is administered orally at a dosage of about 50 mg BID, about 100 mg BID, about 150 mg BID, about 200 mg, about 225 mg, about 250 mg, about 275 mg, about 300 mg, about 350 mg, about 400 mg, about 450 mg, about 500 mg, about 550 mg, about 600 mg, about 650 mg, or about 700 mg BID, or about 800 mg, or about 900 mg, or about 1100 mg, or about 1200 mg. In some embodiments, Compound A or Compound D is administered orally at a dosage of about 50 mg BID, about 100 mg BID, or about 150 mg BID.

In one embodiment, Compound B or a pharmaceutically acceptable salt thereof, is administered orally. In some embodiments, Compound B or a pharmaceutically acceptable salt thereof, is administered orally at a dosage of about 50 mg BID, about 100 mg BID, about 150 mg BID, about 200 mg, about 250 mg, about 300 mg, about 350 mg, about 400 mg, about 450 mg, about 500 mg, about 550 mg, about 600 mg, about 650 mg, about 700 mg BID, about 800 mg, about 900 mg, about 1100 mg, about 1200 mg, about 1300 mg, about 1400 mg, about 1500 mg, or about 1600 mg, about 1700 mg, or about 1800 mg. In other embodiments, Compound B or a pharmaceutically acceptable salt thereof, is administered orally at a dosage of about 200 mg, about 600 mg, or about 800 mg, or about 900 mg, or about 1200 mg. In some embodiment, Compound B or a pharmaceutically acceptable salt thereof, is administered orally at a dosage of about 200 mg, or about 600 mg.

### Pharmaceutical Compositions

Compound A and Compound B, and Compound D and Compound B can each be administered or provided as the neat chemical, but it is typical, and preferable, to administer or provide the compounds in the form of a pharmaceutical composition or formulation. Accordingly, provided are pharmaceutical compositions that include Compound A and/or Compound B, or Compound D and/or Compound B and a biocompatible pharmaceutical vehicle (e.g., carrier, adjuvant, and/or excipient). The composition can include the compounds as the sole active agent(s) or in combination with other agents, such as oligo- or polynucleotides, oligo- or polypeptides, drugs, or hormones mixed with one or more pharmaceutically acceptable vehicles. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

Compound A and Compound B, or Compound D and Compound B or pharmaceutically acceptable salts thereof, may be administered in the same or separate formulations. In certain embodiments, Compound A or Compound D or a pharmaceutically acceptable salt thereof is present in a pharmaceutical composition comprising Compound A or Compound D or a pharmaceutically acceptable salt thereof, and at least one pharmaceutically acceptable vehicle. In certain embodiments, Compound B or a pharmaceutically acceptable salt thereof is present in a pharmaceutical composition comprising Compound B or a pharmaceutically acceptable salt thereof, and at least one pharmaceutically acceptable vehicle. In one embodiment, the active ingredients (e.g., Compound A and Compound B, or Compound D and Compound B or pharmaceutically acceptable salts thereof) are administered in separate unit dosages (e.g., in separate tablets, pills or capsules, or by different injections in separate syringes).

Techniques for formulation and administration of pharmaceutical compositions can be found in Remington's Pharmaceutical Sciences, 18th Ed., Mack Publishing Co, Easton, Pa., 1990; and Modern Pharmaceutics, Marcel Dekker, Inc. 3rd Ed. (G. S. Banker & C. T. Rhodes, Eds.). The pharmaceutical compositions described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions can be formulated to contain suitable pharmaceutically acceptable vehicles, which may include, for example, inert solid diluents and fillers, diluents, including sterile aqueous solution and various organic solvents, permeation enhancers, solubilizers and adjuvants. For example, the pharmaceutical compositions may comprise pharmaceutically acceptable carriers, and optionally can comprise excipients and auxiliaries that facilitate processing of Compound A, Compound B, or both Compound A and Compound B into preparations that can be used pharmaceutically. In another example, the pharmaceutical compositions may comprise pharmaceutically acceptable carriers, and optionally can comprise excipients and auxiliaries that facilitate processing of Compound D, Compound B, or both Compound D and Compound B into preparations that can be used pharmaceutically. The mode of administration generally determines the nature of the carrier. For example, formulations for parenteral administration can include aqueous solutions of the active compounds in water-soluble form. Carriers suitable for parenteral administration can be selected from among saline, buffered saline, dextrose, water, and other physiologically compatible solutions. In one embodiment, carriers for parenteral administration include physiologically compatible buffers such as Hanks's solution, Ringer's solution, or physiologically buffered saline. For tissue or cellular administration, penetrants appropriate to the particular barrier to be permeated are used in the formulation. Such penetrants are generally known in the art. For preparations including proteins, the formulation can include stabilizing materials, such as polyols (e.g., sucrose) and/or surfactants (e.g., nonionic surfactants), and the like.

Alternatively, formulations for parenteral use can include dispersions or suspensions of Compound A, Compound B, or both Compound A and Compound B prepared as appropriate oily injection suspensions. Similarly, formulations for parenteral use can include dispersions or suspensions of Compound D, Compound B, or both Compound D and Compound B prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing Compound A, Compound B, or both Compound A and Compound B also can be employed for parenteral administration. Liposomes containing Compound D, Compound B, or both Compound D and Compound B also can also be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

In some embodiments, Compound A, Compound B, or both Compound A and Compound B, or the composition thereof, may be formulated for oral administration using pharmaceutically acceptable carriers well known in the art. In other embodiments, Compound D, Compound B, or both Compound D and Compound B, or the composition thereof, may be formulated for oral administration using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

In some embodiments, oral formulations include tablets, dragees, and gelatin capsules. These preparations can contain one or more excipients, which include, for example:

(i) diluents, such as microcrystalline cellulose and sugars, including lactose, dextrose, sucrose, mannitol, or sorbitol;

(ii) binders, such as sodium starch glycolate, croscarmellose sodium, magnesium aluminum silicate, starch from corn, wheat, rice, potato, etc.;

(iii) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;

(iv) disintegrating or solubilizing agents such as cross-linked polyvinyl pyrrolidone, starches, agar, alginic acid or a salt thereof, such as sodium alginate, or effervescent compositions;

(v) lubricants, such as silica, talc, stearic acid or its magnesium or calcium salt, and polyethylene glycol;

(vi) flavorants and sweeteners;

(vii) colorants or pigments, e.g., to identify the product or to characterize the quantity (dosage) of active compound; and

(viii) other ingredients, such as preservatives, stabilizers, swelling agents, emulsifying agents, solution promoters, salts for regulating osmotic pressure, and buffers.

Gelatin capsules may include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragee cores may be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

Provided herein are also unit dosage forms of Compound A and Compound B, or Compound D and Compound B, or pharmaceutically acceptable salts thereof. As used herein, “unit dosage form” refers to physically discrete units, suitable as unit dosages, each unit containing a predetermined quantity of active ingredient, or compound which may be in a pharmaceutically acceptable carrier. One of skill in the art would recognize that the unit dosage form may vary depending on the mode of administration. In one embodiment, the unit dosage form of Compound A and Compound B, or Compound D and Compound B or pharmaceutically acceptable salts thereof, are tablets.

Exemplary unit dosage levels of Compound A or Compound D, or a pharmaceutically acceptable salt thereof, for a human subject may be between about 0.01 mg to about 1000 mg, or between about 50 mg to about 200 mg, or about 25 mg, about 50 mg, about 75 mg, about 100 mg, about 125 mg, or about 150 mg, or about 175 mg, about 200 mg, or about 250 mg.

Exemplary unit dosage levels of Compound B, or a pharmaceutically acceptable salt thereof, for a human subject may be between about 0.01 mg to about 1600 mg, or between about 50 mg to about 200 mg, or about 25 mg, about 50 mg, about 75 mg, about 100 mg, about 125 mg, or about 150 mg, or about 175 mg, about 200 mg, about 250 mg, about 300 mg, about 350 mg, about 400 mg, about 600 mg, about 900 mg, or about 1200 mg.

Compound A, Compound B, or Compound D or pharmaceutically acceptable salts thereof may be administered as one or more unit dosage forms. For example, in one embodiment, a dose of 100 mg of Compound A or Compound D may be orally administered to a subject (e.g., a human subject) in one 100 mg tablet. In one embodiment, a dose of 200 mg of Compound B may be orally administered to a subject (e.g., a human subject) in one 200 mg tablet. In another embodiment, a dose of 600 mg of Compound B may be orally administered to a subject (e.g., a human subject) in three 200 mg tablets.

### Articles of Manufacture and Kits

Compositions (including, for example, formulations and unit dosages) comprising Compound A or Compound D or a pharmaceutically acceptable salt thereof and compositions comprising Compound B can be prepared and placed in an appropriate container, and labeled for treatment of an indicated condition. Accordingly, provided is also an article of manufacture, such as a container comprising a unit dosage form of Compound A or Compound D and a unit dosage form of Compound B, and a label containing instructions for use of the compounds. In some embodiments, the article of manufacture is a container comprising (i) a unit dosage form of Compound A or Compound D and one or more pharmaceutically acceptable carriers, adjuvants or excipients; and (ii) a unit dosage form of Compound B and one or more pharmaceutically acceptable carriers, adjuvants or excipients. In one embodiment, the unit dosage form for both Compound A and Compound B is a tablet. In another embodiment, the unit dosage form for both Compound D and Compound B is a tablet.

Kits also are contemplated. For example, a kit can comprise unit dosage forms of Compound A and Compound B, or Compound D and Compound B, or pharmaceutically acceptable salts thereof, and a package insert containing instructions for use of the composition in treatment of a medical condition. In some embodiments, the kits comprises (i) a unit dosage form of Compound A or Compound D and one or more pharmaceutically acceptable carriers, adjuvants or excipients; and (ii) a unit dosage form of Compound B and one or more pharmaceutically acceptable carriers, adjuvants or excipients. In one embodiment, the unit dosage form for both Compound A and Compound B is a tablet. In another embodiment, the unit dosage form for both Compound D and Compound B is a tablet. The instructions for use in the kit may be for treating a cancer, including, for example, a hematologic malignancy. In some embodiments, the instructions for use in the kit may be for treating cancer, such as leukemia or lymphoma, including relapsed and refractory leukemia or lymphoma. In certain embodiments, the instructions for use in the kit may be for treating acute lymphocytic leukemia (ALL), B-cell ALL, acute myeloid leukemia (AML), chronic lymphocytic leukemia (CLL), small lymphocytic lymphoma (SLL), multiple myeloma (MM), non-Hodgkin's lymphoma (NHL), indolent NHL (iNHL), mantle cell lymphoma (MCL), follicular lymphoma, Waldenstrom's macroglobulinemia (WM), B-cell lymphoma, or diffuse large B-cell lymphoma (DLBCL). In one embodiment, the instructions for use in the kit may be for treating non-Hodgkin's lymphoma (NHL) or chronic lymphocytic leukemia (CLL). In certain embodiments, conditions indicated on the label can include, for example, treatment of cancer.

### Example 1

### Combination of Compound A and Compound B on CLL Cells

Given the importance of microenvironmental signals in the ability of malignant cells to survive, proliferate, and resist standard therapies, the effect of combining a PI3Kδ inhibitor and a Syk inhibitor for use on CLL cells in the presence of stroma-conditioned media was investigated. In this Example, CLL patient samples were assessed for growth inhibition, chemokine release, and pathway activation status using a tetrazolium-based MTS assay, ELISA, and flow cytometry, respectively. Peripheral blood mononuclear cells (PBMCs) were isolated from primary patient samples using a Ficoll-Paque gradient. Compound A (a PI3Kδ inhibitor) and Compound B (a Syk inhibitor) were plated either alone in concentrations known to span their IC₅₀ (0.6 nM to 10 uM), or in combination using equimolar concentrations of each compound (0.6 nM to 10 uM) in 96-well plates. 5×10⁵ primary cells were cultured in triplicate in HS-5 stromal cell conditioned media and growth inhibition was determined after 72 hours.

To explore potential additive, synergistic, or antagonistic interactions between Compound A and Compound B, dose response and interaction indices were calculated using R (Lee et al. 2007) Immunoblotting and flow cytometry were used to measure the inhibitory effects of each compound either alone or in combination on downstream targets known to be associated with BCR activation, including AKT, ERK, and S6 in Ramos cells, a Burkitt's lymphoma cell line, and in primary CLL cells.

Of the 14 primary CLL samples treated with the single agent Compound B, the median cell viability IC₅₀ for all samples was 3.7 μM and an IC₅₀ of ≦2 μM was obtained in 7 samples (50%). Interaction indices were calculated from combination studies using Compound A and Compound B in 7 samples. At least one significantly synergistic concentration of the combination of Compound A and Compound B was found in five of the seven samples. The two remaining CLL samples showed additive responses when treated with the combination. Patient samples cultured in the presence of conditioned media resulted in increased CCL2, CCL3, and CCL4 levels. Production of these chemokines by CLL cells was reduced by both Compound A and Compound B, alone and in combination. Furthermore, treatment with individual inhibitors decreased S6 and ERK, phosphorylation, an effect further enhanced by the combination of PI3Kδ and Syk inhibition.

Thus, the results from this Example indicate that both PI3Kδ and Syk inhibition reduces CLL survival. Dual targeting can also induce synergistic growth inhibition and further disrupt chemokine signaling. Given the complexity of BCR signaling pathways, simultaneous targeting of multiple kinases has the potential to significantly increase clinical activity. Since inhibition of BCR mediated kinases has demonstrated good patient tolerability, combination therapy targeting both PI3Kδ and Syk may provide a novel treatment approach, especially in patients with poor risk disease that may not respond optimally to single agents.

### Example 2

### Evaluation of the Safety, Pharmacokinetics and Pharmacodynamics of Compound A and Compound B, when Administered Alone or in Combination in Humans

In this Example, the safety, pharmacokinetics (PK), pharmacodynamics (PD) of Compound B given alone or together with Compound A were evaluated in healthy human subjects.

Treatment Plan and Regimen

Compound B and Compound A were each given twice-daily (BID) with food for 4 days (Day 4: AM dose only) either alone or together. Three groups of healthy individuals received either 200 mg of Compound B and 100 mg of Compound A, 600 mg of Compound B and 100 mg of Compound A, or 600 mg of Compound B and 150 mg of Compound A. Both compounds were administered orally twice daily (BID).

Compound B was provided as 25-, 100-, and 200-mg strength tablets. Compound A was provided in tablets intended for oral administration. Each tablet contained 100 or 150 mg of active Compound A.

For each cohort, the treatment schedule for each cohort was as follows: days 1-4: treatment with Compound B BID under fed conditions; days 5-14: observation; days 15-18: treatment with Compound A BID under fed conditions; days 19-28: observation; days 29-32: treatment with Compound A and Compound B BID under fed conditions; and days 33-42: observation.

Dose-escalation by cohort and continuation of dosing within a cohort was governed based on safety and tolerability. In each cohort, all 8 subjects received Compound B and Compound A first alone and then in combination. All doses were given under fed conditions.

A10 doses of oral study drugs were administered twice daily at approximately the same time. The standardized meal was consumed within 30 minutes or less. After consuming the standardized meal, treatment occurred within 5 minutes. No food was consumed for approximately 4 hours post dose, and a standardized lunch meal (about 500 calories and about 13 g fat) was provided after sample collections.

Pharmacokinetics (PK), Pharmacodynamics (PD) and Safety

PK and PD were assessed for functional inhibition of ex vivo αIgE-stimulated CD63 expression on basophils. Safety was assessed throughout the study.

Pharmacokinetics

On Day 1, PK samples were collected at pre-dose, 2, 4, 8, and 12 hours post dose. On Day 4, samples were collected relative to the AM dose of study drug(s) at the following time points: pre-dose, 1, 2, 2.5, 3, 4, 6, 8, 10, 12, 18, 24, 36, and 48 hours post dose. Trough PK samples were collected on Day 3 prior to dosing.

The following plasma PK parameters were calculated for Compound B and Compound A: Cₘₐₓ, Tₘₐₓ, Cₗₐₛₜ, Tₗₐₛₜ, AUC₀₋ₗₐₛₜ, AUCₜₐᵤ, λ_{z}, T_{1/2}, CL/F, and V_{z}/F. In addition, metabolites of Compound B and Compound A were also evaluated.

The primary PK endpoints of this study was the characterization of the single dose and steady-state plasma PK parameters of Compound B and Compound A administered alone and in combination.

Pharmacodynamics

On Day 1, PD samples were collected at pre-dose, 2, 4, 8, and 12 hours post dose. On Day 4, PD samples were collected at pre-dose, 2, 4, 8, 12, 24, and 48 hours post dose. PD samples were collected on Day 3 prior to dosing (at the same time PK samples were collected).

The following blood PD parameters were measured: # cells, # of CD123⁺/HLA⁻ cells (basophils), # CD63⁺ basophils, phospho-SYK (Y₅₂₅), and phospho-AKT. The calculated parameters are % CD63⁺ basophils, % phospho-SYK (Y₅₂₅), and phospho-AKT versus positive controls. In addition, downstream markers such as activated BTK and activated TORC1/2 were also evaluated.

Relationships between Compound B and Compound A in PK and PD activity were also explored. Correlation of Compound B and Compound A exposure (as assessed by PK parameter estimates including Cₜₐᵤ, Cₘₐₓ, and AUC) with PD activity (as assessed by CD63, pSYK and pAKT) was also evaluated.

Safety

Safety and tolerability of Compound B and Compound A when administered alone and in combination was assessed. The endpoints evaluated include AEs, physical examination and clinical laboratory test findings, 12-lead ECG abnormalities and interval measurements, and vitals signs measurements.

Safety data for each cohort was reviewed after the 10-day observation period following each 4 day treatment period. Subsequent treatments in each cohort were only initiated following post dose safety assessments.

### Results

In general, adverse events (AEs) were mild to moderate: some individual reported headache and somnolence and one individual discontinued treatment due to Grade 1 ALT/AST elevation during Compound A 150 mg dosing. As shown in Table 3, the exposure of Compound B were higher with that of Compound A (51% to 64% for AUCₜₐᵤ; 74% to 96% for Cₜₐᵤ) and exposure of Compound A had minor variations. Compound B (600 mg) plus Compound A (≧100 mg) dosing provided near complete inhibition of CD63 expression over the dosing interval. The results are consistent with the unexpected synergistic results in Examples 1 and 2.

TABLE 1
PK Data Collected from Administration of Compound B
and Compound A, Combined versus Alone

| | | | % Geometric |
|---|---|---|---|
| | | | Least |
| | | | Squares Means |
| PK | | | Ratio |
| Parameter | Mean (% CV) | | (90% CI) |
| Com- | Compound B | Compound B 200 mg + | |
| pound | 200 mg | Compound A 100 mg | Combination vs. |
| B | (N = 8) | (N = 8) | Alone |
| AUCₜₐᵤ | 4150 (48) | 6750 (47) | 1.64 (1.41, 1.91) |
| Cₘₐₓ | 618 (40) | 915 (38) | 1.50 (1.24, 1.80) |
| Cₜₐᵤ | 201 (661) | 380 (62) | 1.96 (1.61, 2.38) |
| | | Compound B 600 mg + | |
| Com- | Compound B | Compound A | |
| pound | 600 mg | 100/150 mg | |
| B | (N = 16) | (N = 14) | |
| AUCₜₐᵤ | 12700 (69) | 16000 (55) | 1.51 (1.30, 1.75) |
| Cₘₐₓ | 1570 (60) | 1900 (55) | 1.38 (1.20, 1.59) |
| Cₜₐᵤ | 832 (82) | 1120 (72) | 1.74 (1.48, 2.03) |
| | | Compound A 100 + | |
| Com- | Compound A | Compound B 200 or | |
| pound | 100 mg | 600 mg | |
| A | (N = 16) | (N = 16) | |
| AUCₜₐᵤ | 8070 (22) | 9510 (25) | 1.17 (1.13, 1.22) |
| Cₘₐₓ | 1660 (18) | 1880 (18) | 1.13 (1.07, 1.20) |
| Cₜₐᵤ | 127 (44) | 170 (50) | 1.33 (1.20, 1.48) |
| Com- | Compound A | Compound A 150 mg + | |
| pound | 150 mg | Compound B 600 mg | |
| A | (N = 7) | (N = 6) | |
| AUCₜₐᵤ | 13800 (15) | 15800 (20) | 1.11 (1.00, 1.24) |
| Cₘₐₓ | 2680 (11) | 2870 (21) | 1.06 (0.93, 1.21) |
| Cₜₐᵤ | 320 (43) | 411 (29) | 1.23 (1.03, 1.47) |

Units for AUCₜₐᵤ: ng.h/ml;
Cₘₐₓ/Cₜₐᵤ: ng/ml; all doses BID

Based on the results in this Example, Compound B and Compound A were generally well-tolerated over the dose range evaluated. Administration of Compound B and Compound A in combination provided substantial PD response over the dosing interval.

### Example 3

### Inhibition of Anti-IgE-Stimulated CD63 Expression in Human Whole Blood Basophils

This example evaluated the in vitro activity for inhibition of anti-IgE (αIgE)-stimulated CD63 expression in human whole blood basophils from 3 normal donors.

Compounds A and B were prepared as 10 mM stocks in dimethyl sulfoxide (DMSO). In this Example, the compounds were either thawed from 10 mM DMSO stocks frozen in 0.75 mL polypropylene tubes at −20° C., or aliquoted from 10 mM DMSO stocks stored at room temperature in glass storage vials.

CD63 Assay in Human Whole Blood Basophils:

Human whole blood was collected in heparinized vacutainers and used within 6 hours of collection. Compound B and Compound A were diluted and added into blood as described below and 100 μL was incubated in a 96-deep well V-bottom plate for one hour at 37° C. Following the incubation, 20 μL of potentiation buffer B from the Basotest kit was added to each well and incubated an additional 10 minutes at 37° C. Subsequently, the FcεR was crosslinked by the addition of 100 μL of αIgE to a final concentration of 10 μg/mL and incubated 20 minutes at 37° C. Degranulation was arrested by placing the plate on ice for five minutes. Cells were stained with 20 μL of a staining cocktail containing CD123-PE/CD63-FITC/HLA-DR-Percep and incubated 20 minutes on ice. Two mL of red cell lysis buffer G from Basotest kit was added to each well and incubated at room temperature for ten minutes. The plate was centrifuged at 365×g for 10 mM at 20° C. and the supernatant decanted. The cell pellet was washed with 2 mL of washing solution (Buffer A from Basotest kit) and centrifuged at 365×g for 10 min at 20° C. The cells were resuspended in Buffer A from Basotest kit and kept on ice in a 250 μL 96-well U-bottom plate until read by multicolor flow cytometry on a BD FACS Canto or Calibur instrument.

Drug Combination Assay:

To test the two-compound combinations in a 96-well plate format, 2-fold serial dilutions in 100% DMSO of Compound A (7 concentrations starting at 1 or 5 μM) were combined with 2-fold serial dilutions in 100% DMSO of Compound B (9 concentrations starting at 3 or 10 μM). In addition, the serial dilution of Compound A alone, the serial dilution of Compound B alone, DMSO with αIgE (the 100% degranulation control) and DMSO only (the 0% degranulation control) were tested. Two μL of the dilutions were added to whole blood and processed as described above.

Data Analysis:

The combination effect was determined by the analysis of data from the CD63 assay using MacSynergy II program (University of Michigan, Ann Arbor, Mich.) developed by Prichard and Shipman. See Prichard M N, Shipman C, Jr., Antiviral Res 1990; 14 (4-5):181-205. The software calculates theoretical inhibition assuming an additive interaction between compounds (based on the Bliss Independence model) and quantifies statistically significant differences between the theoretical and observed inhibition values. Plotting these differences in three dimensions results in a surface where elevations in the Z-plane represent synergy and depressions represent antagonism between compounds. The calculated volumes of surface deviations are expressed in μM². %. The program allows for the assessment of the degree of synergy, additivity, or antagonism for the tested pairwise combination and defines the combination effect according to a specific numeric value of combination log volume (μM². %) calculated from the inhibition data. To determine the nature and the degree of the analyzed interactions, combination volume values with a Bonferonni adjustment were calculated at their 95% confidence levels. There were several categories of combination effects: highly antagonistic, slightly antagonistic, additive, slightly synergistic, and highly synergistic. The criteria of each category was summarized in Table 2.

TABLE 2
Compound Combination Effects Defined According to the
Value of Combination Volume (μM² · %)

| Combination volume value [μM² · %] | Combination effect |
|---|---|
| ≧100 | Highly synergistic |
| ≧50 to <100 | Slightly synergistic |
| ≧−50 to <50 | Additive |
| ≧−100 to <−50 | Slightly antagonistic |
| <−100 | Highly antagonistic |

In this Example, Compound B was tested in pairwise combination with Compound A. The compound combination at varying doses was assessed in 3 independent experiments performed in triplicate in whole blood from 3 individual donors. The MacSynergy II program was used to process the data and determine the inhibitory combination effect. The combination volume values of Compound B in the three donor samples were 110, 499, and 183 μM². % for expression of CD63 inhibition in human whole blood, with a mean log volume of 264±267.2 μM². %. In all triplicate samples, the values of the combination log volumes values were more than 100. This suggests that, when combined with Compound A, Compound B exhibited highly synergistic inhibitory effects. Moreover, the combination of Compound B and Compound A showed no sign of antagonism in any sample tested.

TABLE 3
Inhibition of αIgE-Mediated CD63 Expression in Human Whole
Blood Basophils by Compound B and Compound A in Combination

| | Synergy/Antagonism volume [μM² · %] | | | | | Combination |
|---|---|---|---|---|---|---|
| Readout | Type | Exp. 1 | Exp. 2 | Exp. 3 | Mean ± SD^{a} | effect |
| CD63 | Synergy | 109.5 | 499.5 | 183.0 | 264.0 ± 267.2 | Highly |
| | Antagonism | 0.0 | 0.0 | −9.3 | −3.1 ± 5.4 | Synergistic |

^{a}Data represents mean values ± standard deviations from 3 independent experiments performed in triplicate.

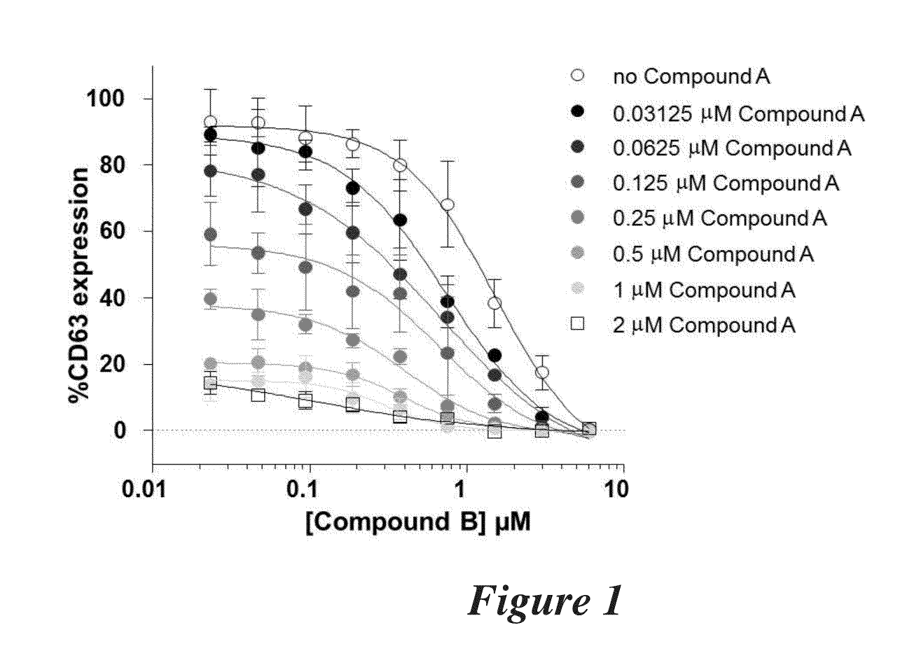

Inhibition curves from the combination compound treatment were generated for each individual donor. A representative combination curve dataset from a single donor is depicted in FIG. 1.

### CONCLUSION

This Example demonstrates that the administration of Compound A and Compound B in combination exhibited highly synergistic in vitro inhibition of CD63 expression in human basophils. In particular, the administration of Compound A and Compound B in combination exhibited synergistic effects at inhibiting CD63 expression following αIgE stimulation based on statistical analysis using a Bonferonni adjusted log volume from three individuals (264.0±267.2 μM². %). Moreover, no antagonism between Compound B and Compound A was detected.

### Example 4

### Inhibition of Phospho-Mek and Phospho-Erk in the B-Cell Lymphoma Cell Line, DHL-4, By Compound B Administered in Combination with Compound A

In this Example, the in vitro activity of Compound B was tested in combination with Compound A for inhibition of anti-IgG (αIgG)-stimulated phosphorylation of phospho-Mek (pMek) and phospho-Erk (pErk) expression in the diffuse large B-cell lymphoma cell line, DHL-4. The administration of Compound A and Compound B in combination exhibited highly synergistic effects at inhibiting both Mek and Erk phosphorylation based on statistical analyses using a Bonferonni adjusted log volume from 3 independent experiments (197.3±125.4 μM². % and 100.2±36.1 μM². % for pErk and pMek respectively).

Compounds:

The compound samples were prepared according to the procedure described in Example 3 above.

Reagents:

The reagents used in this Example were obtained from commercially available sources.

Cell Line:

SU-DHL-4 cell line was obtained from DSMZ (Leibniz-Institut DSMZ—Deutsche Sammlung von Mikroorganismen and Zellkulturen GmbH). DHL-4 were maintained in RPMI-1640 base medium containing 10% FBS, 1% penicillin-streptomycin, 10 mM HEPES and 1% GlutaMax.

Drug Combination Assay:

To test specific 2-drug combinations in a 96-well plate format, 2-fold serial dilutions (in DMSO) of Compound A (7 concentrations starting at 10 mM) were combined with 2-fold serial dilutions (in DMSO) of Compound B (9 concentrations starting at 10 mM). Four μL of the 1000× compound mixture was diluted 1:100 in RPMI-1640 and further diluted 1:10 into the final assay plate. Each combination of two compounds was tested in triplicate (3 identical plates set up in parallel). Controls containing no stimulation or anti-IgG alone were included on each assay plate in DMSO, representing 0% and 100% signal.

Meso Scale Assays:

DHL-4 cells grown in log phase were starved in RPMI-1640 base medium containing 1% penicillin-streptomycin and 10 mM HEPES at 6×106 cells/mL for 1 hour at 37° C. and 5% CO2. Cells were seeded at 2-5×105 cells per well in 96-well assay plates with additional medium (RPMI-1640). Compound was added to the cells for 1 hour at 37° C. and 5% CO2. Cells were stimulated for 5 minutes with αIgG (10 μg/mL). Cells were centrifuged at 300×g for 5 minutes at room temperature, supernatants were discarded, and cell pellets were lysed in 50 μL 1×RIPA buffer on ice for 10 minutes. Lysates were then either immediately assayed in Meso Scale (MSD) plates or frozen at −80° C. until use. The pErk and pMek assays were performed as per manufacturer's instructions. The plates were immediately read on the Meso Scale Sector 2400 Imager (Meso Scale Discovery, Gaithersburg, Md.).

Data Analysis:

The combination effect of each tested pair of inhibitors was determined by the analysis of datasets from the assays of this Example using the software and procedure described in Example 3 above.

In this Example, Compound B was tested in pairwise combination with Compound A. The compound combination was assessed in 3 independent experiments performed in triplicate in DHL-4 cells. MacSynergy II program was used to process the data and determine the inhibitory combination effect. In all three independent experiments, the values of combination volumes (μM². %) for pErk and pMek were 197.3±125.4 μM². % and 100.2±36.1 μM². % (mean Boneferoni adjusted log volume), respectively. The results suggest that, when combined with Compound A, Compound B was observed to exhibit highly synergistic inhibitory effects that was unexpected. See Table 4. Moreover, the combination of Compound B and Compound A showed no sign of antagonism in any sample tested.

TABLE 4
Inhibition of αIgG-Mediated pMek and pErk in DHL-4
Cells by Compound B and Compound A in Combination

| | Synergy/Antagonism volume [μM² · %] | | | | | Combination |
|---|---|---|---|---|---|---|
| Readout | Type | Exp. 1 | Exp. 2 | Exp. 3 | Mean ± SD^{a} | effect |
| pMek | Synergy | 139.3 | 93.1 | 68.2 | 197.3 ± 125.4 | Highly |
| | Antagonism | 0.0 | −4.5 | 0.0 | −1.5 ± 2.6 | Synergistic |
| pErk | Synergy | 74.6 | 325.2 | 192.0 | 100.2 ± 36.1 | Highly |
| | Antagonism | −8.0 | 0.0 | 0.0 | −2.7 ± 4.6 | Synergistic |

^{a}Data represents mean values ± standard deviations from 3 independent experiments performed in triplicate.

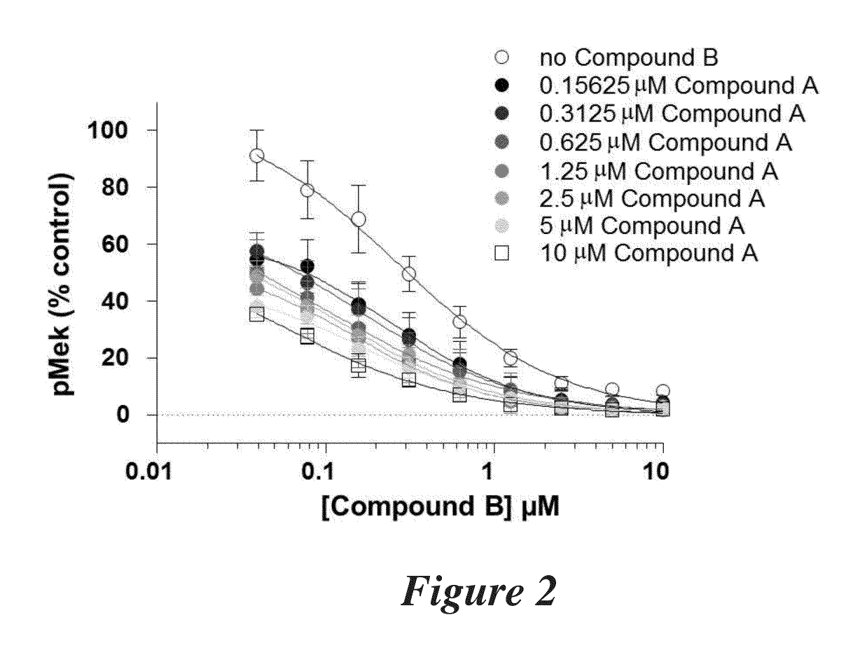

Inhibition curves from the combination drug treatment were generated for pMek and pErk in DHL-4 cells. A representative combination pMek curve dataset is depicted in FIG. 2.

This Example demonstrates that the administration of Compound A and Compound B in combination exhibited highly synergistic in vitro inhibition of pMek and pErk expression in DHL-4 cells following αIgG stimulation of the BCR. Moreover, no antagonistic interactions between Compound B and Compound A was detected.

### Example 5

### Inhibition of Chemokine Release in Malignant B-Cell Lines by Compound B Administered in Combination with Compound A

In this Example, the in vitro activity of Compound B was tested in combination with Compound A for inhibition of release of chemokines CCL3 and CCL4 from 3 distinct human B-cell malignant cell lines: DHL4 (a Diffuse Large B-cell Lymphoma line), Mino (a Mantle Cell Lymphoma line), and CCRF-SB (a B-Cell Acute Lymphoblastic Leukemia line). Synergy evaluations were based on statistical analysis using a Bonferonni adjusted log volume from 3 independent experiments. The synergy was observed to be stronger for CCL4 production than CCL3 in all 3 cell lines tested. The strongest synergy was detected for the drug combination in the DHL-4 cell line and resulted in a CCL4 mean Bonferonni adjusted log volume of 1671.7±1228.1 μM². % (n=3). The weakest synergy was observed for CCL3 production in the CCRF-SB cells, where the mean Bonferonni adjusted log volume was 88.0±66.8 μM². %.

Compounds:

The compound samples were prepared according to the procedure described in Example 3 above.

Reagents:

The reagents used in this Example were obtained from commercially available sources.

Cell Lines:

SU-DHL-4 (DHL-4) cells were obtained from DSMZ (Leibniz-Institut DSMZ—Deutsche Sammlung von Mikroorganismen and Zellkulturen GmbH) and Mino, CCRF-SB, and M2-10B4 cell lines were obtained from ATCC (American Tissue Type Collection). All cell lines were maintained in growth medium (RPMI-1640 base medium containing 10% FBS, 1% penicillin-streptomycin, 10 mM HEPES and 1% GlutaMax).

Drug Combination Assay:

CCL3 and CCL4 levels were determined using an AlphaLISA assay (Perkin-Elmer, Shelton, Conn.). To test specific 2-drug combinations in a 96-well plate format, 2-fold serial dilutions in 100% DMSO of Compound A (7 concentrations starting at 10 μM, in the vertical top to bottom direction pipetted into rows A to G, columns 1 to 10) were combined with 2-fold serial dilutions in 100% DMSO of Compound B (9 concentrations starting at 5 μM in the horizontal right to left direction pipetted into columns 10 to 2, rows A to H). For each drug alone the serial dilution of Compound A in column A did not contain Compound B and the serial dilution of Compound B in row H did not contain Compound A. Column 11 contained only DMSO (the 100% chemokine release control) and column 12 contained 100 mM (1000×) of staurosporine (the 0% chemokine release control). Each well of the final prepared compound serial dilution plate contained a total volume of 4 μL of DMSO with the drug combinations as described.

Chemokine Release:

The mouse stromal cell line M2-10B4 was plated in 96-well assay plates at a cell density of 1.0×10⁴ cells per well in 100 μL of growth medium. Assay plates were incubated at 37° C. in a 5% CO₂ incubator for 48 hours to form a confluent monolayer. Media was removed by gently pipetting the medium off the attached stromal cells. Suspension cell lines DHL-4, Mino, and CCRF-SB were washed once in growth medium and plated on top of the stromal layer at a cell density of 5×10⁴ cells per well in 135 μL of growth medium in triplicate plates. The compound serial dilution plate, as described above with 4 μL per well, was resuspended in 196 μL of RPMI-1640 without additives (a 1:200 dilution of each compound) and 15 μL of this dilution was added to the 135 μL containing the B and stromal cells. Assay plates were incubated at 37° C. in a 5% CO₂ incubator for 18-24 hours and centrifuged at 300×g for 5 minutes at room temperature and 75 μL of supernatant from each well was removed. Supernatants were either immediately assayed with the AlphaLISA detection system, or stored at −80° C. for 1-2 days in a 96-well V-bottom plate covered with foil until use.

AlphaLisa Detection:

AlphaLISA assays were performed according to the manufacturer's instructions. The high sensitivity protocol was performed with 5 μL of supernatant from the assay wells. CCL3 and CCL4 release was detected with a Paradigm SpectraMax Instrument (Molecular Devices, Sunnyvale, Calif.) with an AlphaLISA detection cartridge.

Data Analysis:

The combination effect of each tested pair of inhibitors was determined by the analysis of datasets from the assays of this Example using the software and procedure described in Example 3 above.

In this Example, Compound B was tested in a pairwise combination with Compound A. The compound combination was assessed in 3 independent experiments performed in triplicate. The experiments were performed in 3 cell lines, representing 3 distinct B-cell malignancies. The MacSynergy II program was used to process the data and determine the inhibitory combination effect. Based on the values of combination log volumes (μM². %), Compound B was observed to exhibit highly synergistic inhibitory effects when combined with Compound A, showing combination log volume values of 1672, 236, and 671 μM². % for CCL4 inhibition in DHL-4, CCRF-SB and Mino cell lines, respectively. See Table 5 below. Similarly, in DHL-4 and Mino cell lines there was a highly synergistic combination effect for CCL3 production with volume values of 975 and 438 μM². %. See Table 5 below. The combination of Compound B and Compound A produced a slightly synergistic inhibition of CCL3 in the CCRF-SB cell line. See Table 5 below. Moreover, the combination of Compound B and Compound A showed no sign of antagonism in any of the cell lines tested.

TABLE 5
Inhibition of CCL3 and CCL4 by Administration
of Compound B and Compound A in Combination

| | | Synergy/Antagonism volume [μM² · %] | | | | | Combination |
|---|---|---|---|---|---|---|---|
| Cell Line | Chemokine | Type | Exp. 1 | Exp. 2 | Exp. 3 | Mean ± SD^{a} | effect |
| DHL-4 | CCL3 | Synergy | 605.2 | 642.4 | 1678.0 | 975.2 ± 609.0 | Highly |
| | | Antagonism | 0.0 | 0.0 | 0.0 | 0.0 | Synergistic |
| CCRF-SB | | Synergy | 148.9 | 98.5 | 16.6 | 88.0 ± 66.8 | Slightly |
| | | Antagonism | 0.0 | 0.0 | −0.3 | −0.1 ± 0.2 | Synergistic |
| Mino | | Synergy | 735.7 | 186.9 | 390.4 | 437.6 ± 277.4 | Highly |
| | | Antagonism | 0.0 | 0.0 | 0.0 | 0.0 | Synergistic |
| DHL-4 | CCL4 | Synergy | 692.1 | 1273.6 | 3049.5 | 1671.7 ± 1228.1 | Highly |
| | | Antagonism | 0.0 | 0.0 | 0.0 | 0.0 | Synergistic |
| CCRF-SB | | Synergy | 295.6 | 316.2 | 390.4 | 235.6 ± 122.2 | Highly |
| | | Antagonism | −2.0 | −1.9 | 0.0 | −1.3 ± 1.1 | Synergistic |
| Mino | | Synergy | 632.5 | 133.3 | 1248.3 | 671.4 ± 558.5 | Highly |
| | | Antagonism | 0.0 | 0.0 | 0.0 | 0.0 | Synergistic |

^{a}Data represents mean values ± standard deviations from 3 independent experiments performed in triplicate.

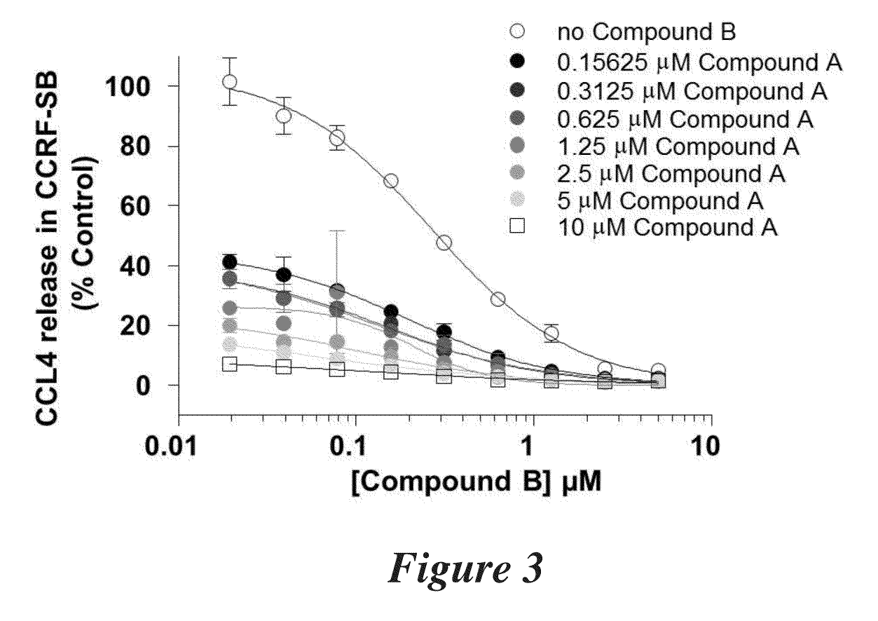

Inhibition curves from the combination drug treatment were generated for each cell line tested. A representative Compound B and Compound A combination curve dataset for inhibition of CCL4 production in CCRF-SB for each cell line are depicted in FIG. 3.

This Example demonstrates that the administration of Compound A and Compound B in combination exhibited highly synergistic in vitro inhibition of CCL3 and CCL4 release from DHL-4 and Mino cell lines co-cultured with a murine stromal cell line, M2-10B4. In addition, a highly synergistic effect was observed in the CCRF-SB cell line for inhibition of CCL4, with a slightly synergistic effect on CCL3 production. Moreover, no antagonistic interactions between Compound B and Compound A were detected.

### Example 6

### Effects of Combining Administration of Compound A and Compound B

This Example demonstrates the synergistic effects of targeting PI3Kδ and Syk together. The administration of both Compound A and Compound B resulted in unexpectedly decrease in CLL cell viability.

Peripheral blood (PB) or bone marrow (BM) was collected from 14 CLL patients with untreated, relapsed, or refractory diseases. Mononuclear cells were isolated using a Ficoll-Paque gradient and were plated in triplicate into 96-well containing HS-5 conditioned media plus equimolar concentrations of Compound A and Compound B (10 μM to 0.61 nM). After 72 hours, viability was determined by MTS assay (CellTiter 96, Promega). R was used to calculate interaction indices and develop 95% confidence intervals to determine synergy.

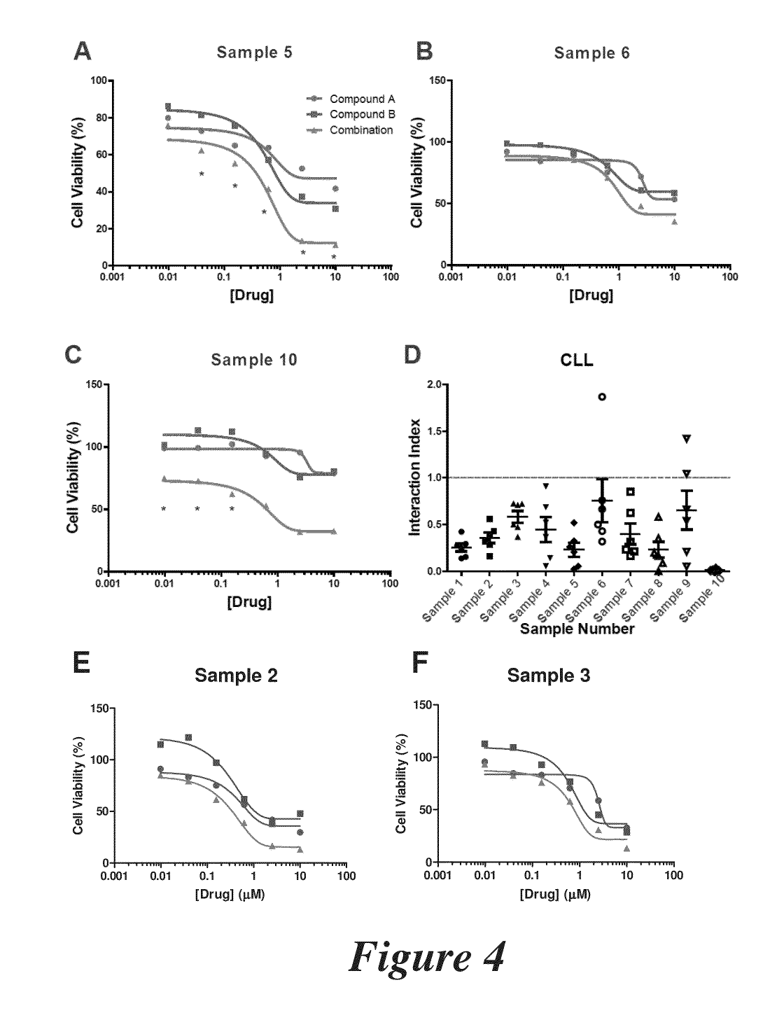

Results of this study were summarized in FIGS. 4A-R. As shown in FIGS. 4A and 4C, as well as other graphs in FIGS. 4E-R, unexpected synergistic effects in CLL samples were observed at several concentrations (e.g. denoted with an asterisks (*) in the figures) of the combination of Compound A and Compound B exhibited. FIG. 4C showed the unexpected synergistic effects detected in a sample isolated from bone marrow that was resistant to each compound alone. In FIG. 4D, the cells treated with both Compound A and Compound B exhibited an increased apoptosis compared to the cells treated to either compound alone. These results suggest that inhibition of both PI3Kδ and Syk reduce the survival and the viability of CLL.

### Example 7

### Inhibition of AKT and S6 Phosphorylation

This Example demonstrates that Compound A and Compound B, alone or in combination, inhibited or decreased signaling downstream of the b-cell receptor (BCR) including phosphorylation of Akt and S6. Moreover, the combination of Compound A and Compound B resulted in unexpectedly reduced or inhibited phosphorylation of S6 when compared to each individual compound alone.

Ficoll purified CLL cells obtained from 15 patients were co-cultured with HS5 stromal cells then treated with each compound alone or in combination. CLL cells were stained with anti-CD5-FITC and either anti-phospho-Akt S473 (Alexa Fluor 488), phospho-S6 or an isotope-matched control antibody (mouse IgG1-Alexa Fluor 488 conjugate, Cell Signaling) and analyzed by 2-color flow cytometry.

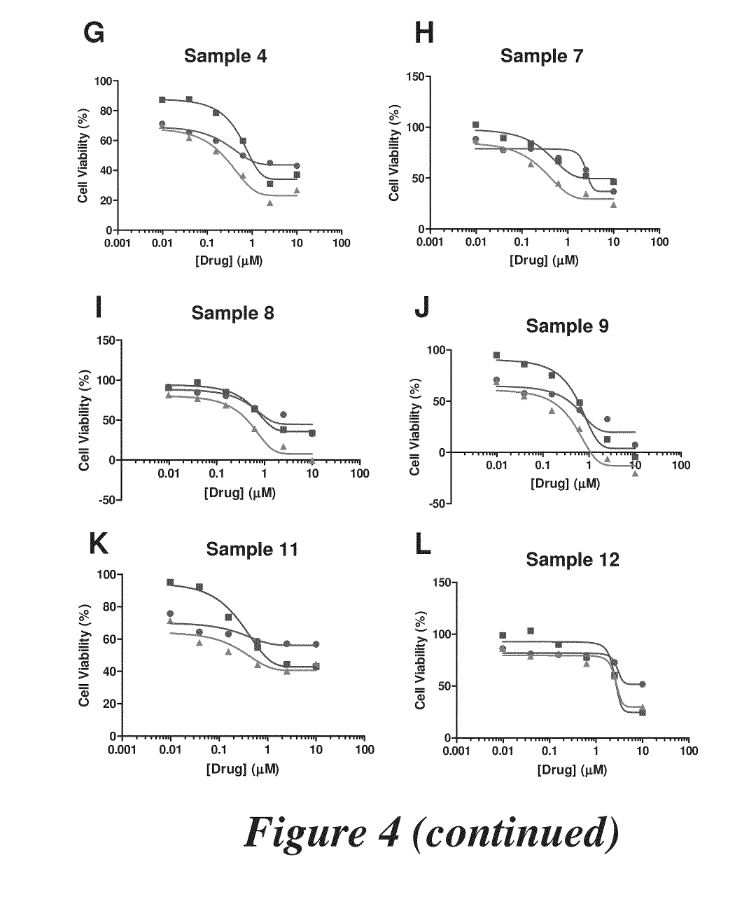

Results were summarized in FIG. 5. As shown in FIG. 5A, the CLL cells treated with both Compound A and Compound B exhibited decreased AKT phosphorylation when compared with the untreated cells. In FIG. 5B, the CLL cells treated with both Compound A and B unexpectedly reduced S6 phosphorylation and the reduction was at a higher level compared to the reduction by either compound alone. Thus, these results showed that the combination of both compounds resulted in unexpected synergistic inhibition of S6 phosphorylation.

### Example 8

### Inhibition of AKT and ERK Phosphorylation

This Example demonstrates that Compound A and Compound B, alone or in combination, inhibited or decreased AKT phosphorylation in Romas cells after IgM mediated b-cell receptor (BCR) stimulation. Also, Compound B alone or in combination with Compound A inhibit or reduced ERK phosphorylation.

5×10⁶ Ramos (a Burkitt's cell line with intact BCR) were plated and treated with 1 uM of Compound A, 1 uM of Compound B, alone or in combination for 25 hours. Twenty μg Goat F(ab′)₂ anti-human IgM (Invitrogen) was added to stimulate the BCR for 30 minutes. Protein were isolated and analyzed using immune-blotting with phospho-Akt (S473), phospho-Erk (T202/Y204), and phospho-S6.

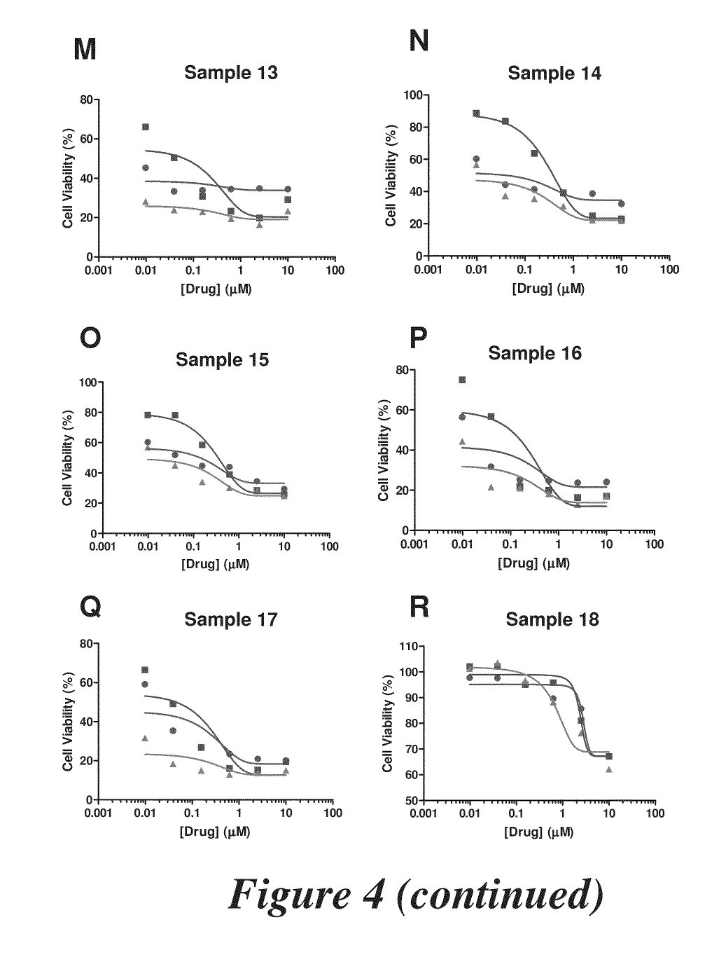

As shown in FIG. 6, the cells treated with either compound or both compounds exhibited near complete inhibition or reduction in AKT phosphorylation. Additionally, the cells treated with Compound B and the combination of both compounds exhibited near complete inhibition or reduction in ERK phosphorylation.

### Example 9

### Inhibition of Chemokine Signaling

This Example demonstrates the effect of Compound A and Compound B, alone or in combination, on CCL2, CCL3, CCL4, and CCL22 secretion after CLL-HS-5 co-culture.

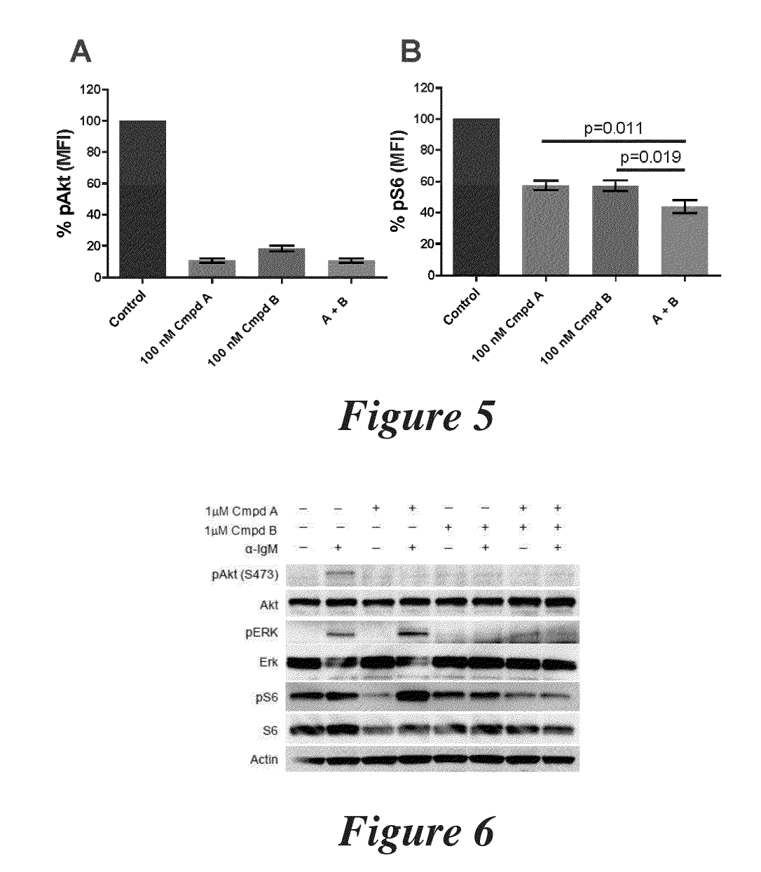

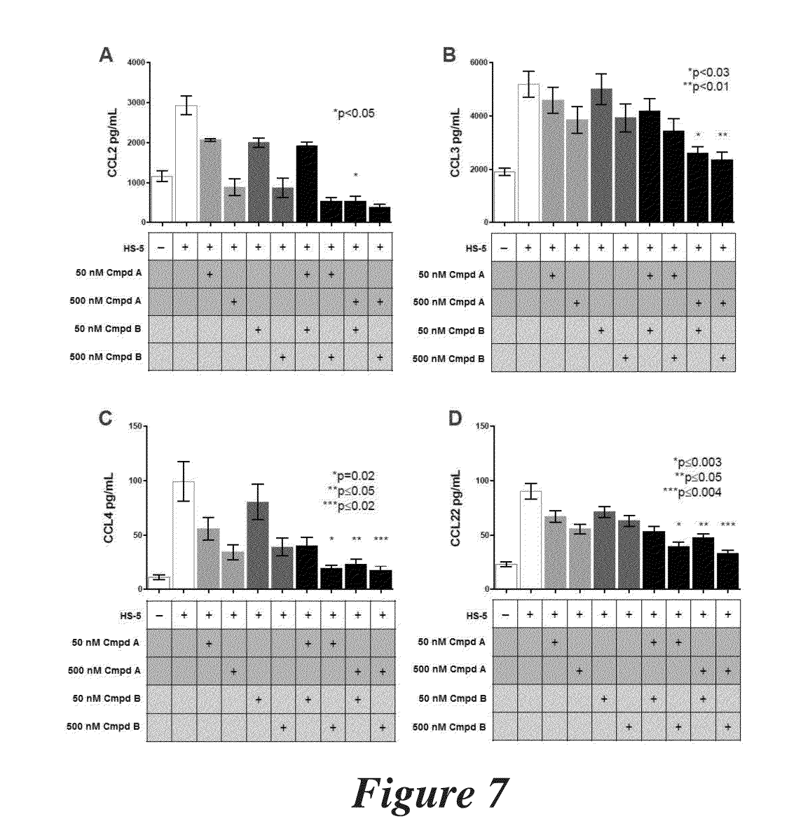

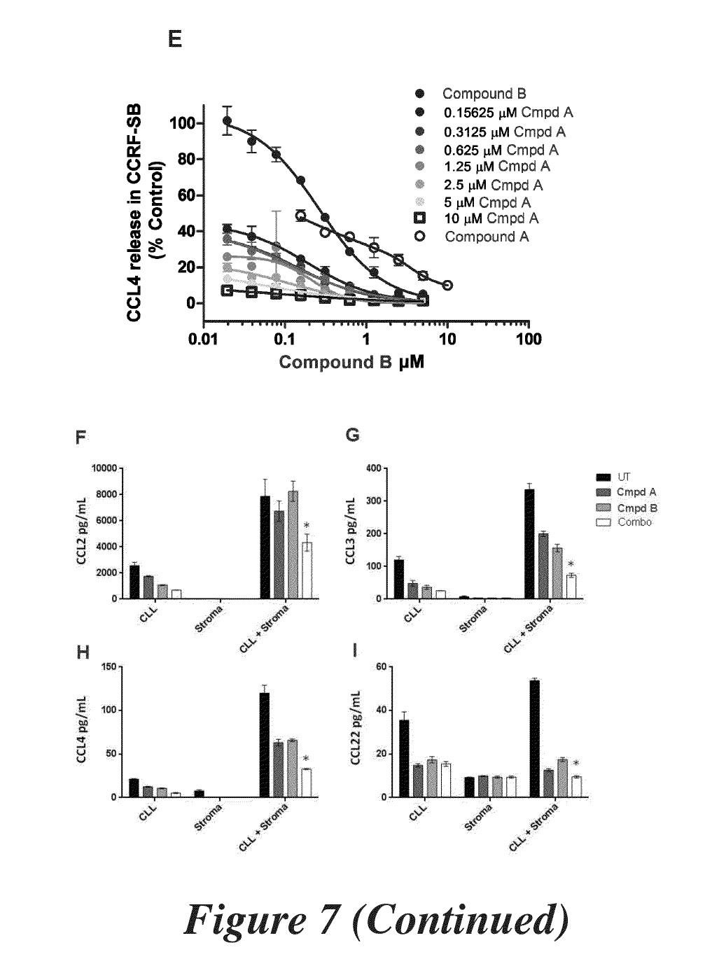

After 24 hours, the supernatants were harvested and assayed for each of the chemokine proteins (−/+HS-5 co-cultures) by quantitative ELISA according to the manufacturer's instructions (Quantikine; R&D Systems). Results were summarized in FIG. 7.

When compared to treatment with each compound alone, the cells treated with the combination of Compound A (500 nM) and Compound B (50 or 500 nM) showed significant (two tailed t-test) decreases in CCL2 (FIG. 7A), CCL3 (FIG. 7B), CCL4 (FIG. 7C) and CCL22 (FIG. 7D).

Also, FIG. 7E showed that, in CRF-SB co-cultures with stromal cells, the treatment with the combination of both compounds resulted in more reduction in CCL4 release. The level of reduction induced by the combination treatment was higher than those treated with either compound alone. Similar levels of reduction as those in the cells treated with the combination of both compounds were detected in the cells treated with Compound B alone at higher concentration resulted.

Also, the effects of Compound A and Compound B, alone or in combination, was tested on CLL cells cultured in primary BM stromal cells. As seen in FIGS. 7F-7I, greater decreases in CCL2, CCL3, CCL4 and CCL22 chemokine levels were observed when Compound A and Compound B were administered in combination. The bar diagram represents the mean (±SEM) supernatant concentrations of CCL2 (F), CCL3 (G), CCL4 (H) and CCL22 (I) assessed at 24 hours.

Increased CCL2, CCL3, CCL4 and CCL22 in CLL and other B-cell malignancies have been correlated with disease severity. Thus, the reduced release of these chemokines in this in vitro study suggest that treating individuals with the combination of both compounds may reduce their disease severity.

### Example 10

### Effect of Compound B on Proapoptotic Activities of Compound A in Primary CLL and iNHL Samples

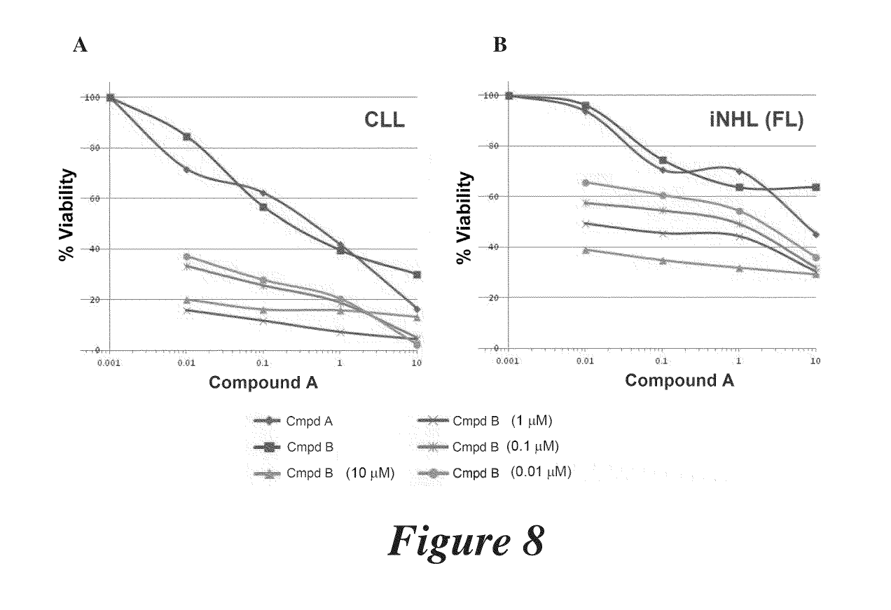

This Example demonstrates the effect of Compound B on proapoptotic activities of Compound A in primary CLL and iNHL samples. The cells were obtained from the peripheral blood of CLL patients and treated with either Compound A, Compound B, or a combination of Compound A and Compound B. The cells were treated with compounds at 0.01, 0.1, 1, and 10 μM. As shown in FIG. 8, CLL and iNHL primary cells treated with the combination of both compounds exhibited lower viability compared to the cells treated with either compound alone. The level of reduction was more than the additive effects of either compound alone, thus the combination of both compounds resulted in unexpected synergistic cell death or apoptosis in CLL and iNHL cells. This synergistic effect was more significant in the primary CLL cells. Additionally, the results in FIG. 8 showed that the synergistic effects increased with the increased concentrations of Compound A and Compound B. The results of this study suggest that the presence of Compound B increases the apoptosis activities induced by Compound A.

### Example 11

### Inhibition of Basophil Activation by Compound A and Compound B

This Example demonstrates the inhibition of ex vivo activation of peripheral blood basophils with anti-IgE, as measured by CD63 expression.

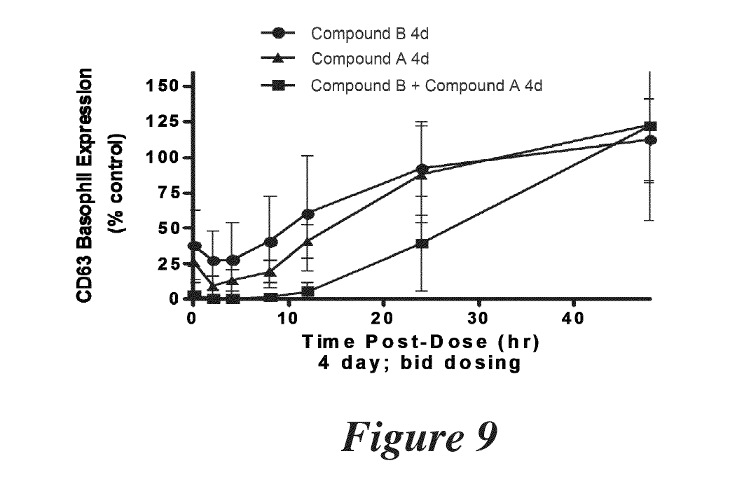

Peripheral blood mononuclear cells (PBMCs) were obtained from subjects from Example 1 before and while they were receiving the study compounds. The ex vivo activation of peripheral blood basophils with anti-IgE, as measured by CD63 expression, was expressed as a percentage of the control pre-study drug level of activation. As shown in FIG. 9, the individuals in Cohorts 2 and 3 who received complete treatment as scheduled exhibited increased inhibition of basophil activation at trough levels when administered with both compounds, compared to administered with either Compound A and Compound B alone.

### Example 12

### Effect of Syk Inhibitor on Sensitivity to Compound A in Resistant Cell Lines

This Example demonstrates that a Syk inhibitor administered before Compound A restores sensitivity or susceptibility to Compound A in resistant a human B-cell line, WSU-FSCCL. The Syk inhibitor used in this Example was Compound C, having the structure:

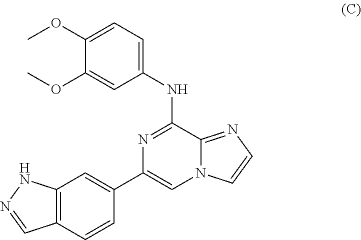 [SMILES: COc1ccc(Nc2nc(-c3ccc4cn[nH]c4c3)cn3ccnc23)cc1OC]

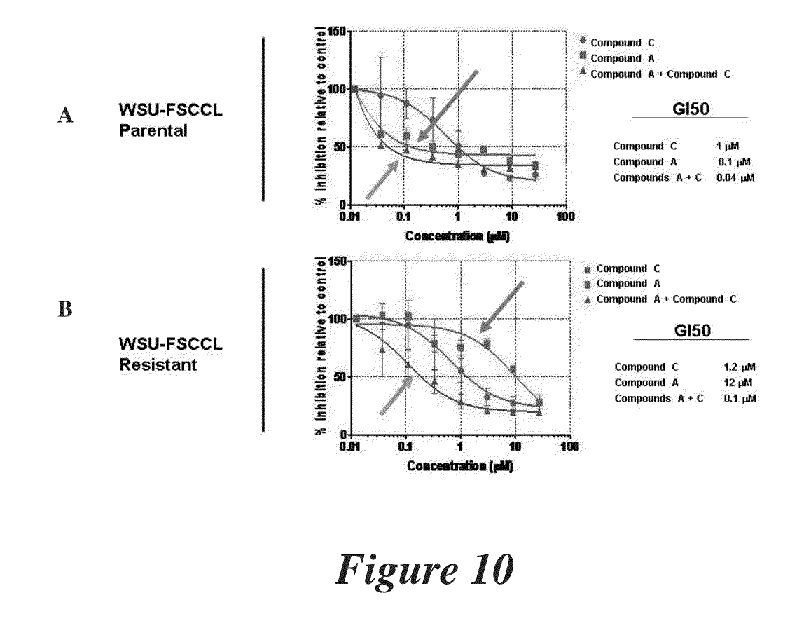

The WSU-FSCCL parental cells were, initially, responsive to treatment of Compound A (FIG. 10A). However, after incubation with the compound for a long time, the WUS-FSCCL parental cells became resistant to the treatment (data not shown). Such cells were cultured and referred to as WSU-FSCCL resistant cells. This reflects a commonly observed clinical situation that patients become resistance or no longer respond to the existing treatment. Both the parental and the resistant cells of WSU-FSCCL were used in this study.

A 7× serial dilution of Compound A and Compound C from a stock of 10 mM (DMSO) was prepared using RPMI+10% FBS (270 μM). The WS-FSCCL cells were added with the compounds in the serial dilution and incubated at 37° C. for 72 hr, followed by the addition of 12.5 μL AlamarBlue (Invitrogen #DAL1100). Plates were then incubated at 37° C. for 4 hours.

The change of color from uptake of the stains in the living cells was measured for absorbance. Fluorescence Ex₅₃₀, Em₅₉₀, Cutoff₅₇₀ on Spectramax M5 (Molecular Dynamics) were determined. To calculate the 50% inhibition of growth (GI50), the reading from the samples were compared to the controls (100% or 0% absorption).

As shown in FIG. 10B, the combination of both compounds was able to remove the resistance or restore the sensitive of WSU-FSCCL resistant cells. Additionally, the resistant cells treated with the combination of both Compound A and Compound C exhibited higher level of reduction in cell viability compared to the cells treated with either Compound A or Compound C alone. This data suggested that the combination of the Syk inhibitor and the PI3K-δ inhibitor resulted in unexpected synergistic effects in reducing cell viability and that the Syk inhibitor has unexpected effects in restoring the sensitivity to the cells in response to the killing or apoptosis induced by the PI3K-δ inhibitor.

### Example 13

### Evaluation of the Efficacy, Safety, Tolerability, and Pharmacodynamics of Compound B in Combination with Compound A in Subjects with Relapsed or Refractory Hematologic Malignancies

In this Example, the efficacy of Compound B given alone or together with Compound A is evaluated in individuals with relapsed or refractory hematologic malignancies. The safety and tolerability of the combination of Compound B and Compound A in individuals with relapsed or refractory hematologic malignancies, the distribution of the maximum tolerated dose levels for the subjects, and the pharmacodynamic measures are also evaluated.

Five cohorts consisting of subjects with indolent non-Hodgkin lymphomas (iNHL), chronic lymphocytic leukemia (CLL), mantle cell lymphoma (MCL), or diffuse large B-cell lymphoma (DLBCL) are studied concurrently. iNHL is studied in 2 cohorts of 40 subjects each: 1 consisting of subjects with follicular lymphoma (FL) and the other consisting of subjects with Lymphoplasmacytoid Lymphoma (LPL), Small Lymphocytic Lymphoma (SLL), and Marginal Zone Lymphoma (MZL)

Compound B and Compound A are administered twice daily (BID) over multiple 28-day cycles in subjects with relapsed or refractory lymphoid malignancies. All subjects receive treatment with Compound B and Compound A BID under fasted conditions. A subject maximum Tolerated Dose Level (sMTD) is determined for each subject using an intra-patient dose escalation scheme. For study purposes, a cycle is considered to be 28 days. In the absence of dose limiting toxicity, dosing continues without interruption. Individuals are evaluated for safety and blood is sampled for exposure and pharmacodynamic monitoring prior to treatment. The treatment is scheduled as following: weekly during Cycle 1, every 2 weeks during Cycle 2, and then once each cycle thereafter. Individuals undergo an assessment to determine their tumor response to treatment every 8 weeks during the first 24 weeks and then every 12 weeks thereafter.

Compound B is available as 200 mg strength tablets. Compound A is available as 150 and 100 mg strength tablets.

To evaluate the biological effects of Compound B, samples for pharmacodynamic assessments of drug activity and resistance are obtained prior to starting treatment, 1.5 and 4.0 hours after first dose, weekly during Cycle 1, every 2 weeks during Cycle 2, on Day 1 of all subsequent cycles and at the time of disease progression.

Serum Cytokines and Chemokines:

Samples are analyzed for disease related systemic cytokines and chemokines, which will include at a minimum, CCL3, CCL4, CXCL12, CXCL13, tumor necrosis factor-α, and C-reactive protein. Initially circulating tumor cells are obtained for pharmcodynamic measurements in subjects with SLL, CLL and MCL only.

Circulating Tumor Cells (CTC)—PhosFlow Analysis:

For SLL, CLL, and MCL subjects, circulating cells are assessed for exploratory biomarkers to evaluate changes in the molecular signature of the malignant cells following treatment with Compound B and to identify mechanisms of resistance to Compound B. These include at a minimum PhosFlow analysis for pAkt, pBlnk, pSyk, pErk, pS6, and pNFkB. Other signaling nodes may also be assessed to evaluate the downstream effects of SYK inhibition and changes that occur in other pathways in response to SYK inhibition.

Circulating Tumor Cells (CTC)—PBMC Analysis:

For SLL, CLL, and MCL subjects only, PBMC samples are assessed using proteomic and molecular approaches to determine at a minimum the phosphorylation status of BTK, Blnk, Pyk2, Mek, ERK, P38, PI3K p85α, PI3Kα, PI3Kβ, PI3Kδ, PI3Kγ, and NFkB; Bcl-2 expression; PTEN mutations and protein expression; and myc protein expression, gene amplification, and translocation.

Circulating Tumor Cells (CTC)—RNA Analysis:

For SLL, CLL, and MCL subjects only, RNA sample assessments include at a minimum expression of c-myc, PTEN, p53, ZAP-70, and bcl-2.

SLL/CLL/MCL Molecular Characterization:

Peripheral blood is collected from SLL, CLL and MCL subjects at screening, on Day 15 of Cycle 2 and at disease progression to assess molecular markers for disease progression, response to treatment and overall prognosis. The baseline status as well as any changes in these molecular markers are correlated with response to Compound B and Compound A.

Calculation of Tumor Control Variables:

Tumor control assessments are based on standardized response and progression criteria for NHL (Cheson et al., J Clin Oncol 2007; 25 (5):579-86) and CLL (Hallek et al. Blood 2008), as specifically modified for this study considering the pharmacology of Compound B and Compound A. The individual and composite endpoints of response and progression (considering changes in lymph node area, liver and spleen size, bone marrow, platelet counts, hemoglobin, neutrophil counts, and peripheral blood lymphocyte counts) are determined Tumor control is documented at each assessment by response category (e.g., CR, PR, SD, definitive PD) as defined for each response parameter, SPD value, percentage change in SPD values from baseline or nadir, date that response is first documented, date that response is confirmed, and date of disease progression.

### Example 14

### Inhibition of Chemokine Release in Malignant B-Cell Lines by Compound B in Combination with Compound D

In this Example, the in vitro activity of spleen tyrosine kinase inhibitor, Compound B, was tested in combination with the phosphoinositide-3-kinase delta inhibitor, Compound D, for inhibition of release of chemokines CCL3 and CCL4 from three distinct human B-cell malignant cell lines: DHL4 (a Diffuse Large B-cell Lymphoma line), Mino (a Mantle Cell Lymphoma line), and CCRF-SB (a B-Cell Acute Lymphoblastic Leukemia line).

Compounds:

Compounds B and D were prepared as 10 mM stocks in DMSO.

Cell Lines:

SU-DHL-4 (DHL-4) cells were obtained from DSMZ (Leibniz-Institut DSMZ—Deutsche Sammlung von Mikroorganismen and Zellkulturen GmbH) and Mino, CCRF-SB, and M2-10B4 cell lines were obtained from ATCC (American Tissue Type Collection). All cell lines were maintained in growth medium (RPMI-1640 base medium containing 10% FBS, 1% penicillin-streptomycin, 10 mM HEPES and 1% GlutaMax).

Combination Assay:

CCL3 and CCL4 levels were determined using an AlphaLISA assay (Perkin-Elmer, Shelton, Conn.). Two-fold serial dilutions in 100% DMSO of Compound D starting at 10 mM were combined with 2-fold serial dilutions in 100% DMSO of Compound B starting at 5 mM. Also, DMSO and 100 μM of staurosporine were used as 100% and 0% chemokine release control, respectively. In CCRF-SB and Mino cells, 10 μM of Compound B was used as 0% chemokine release control.

Chemokine Release:

The mouse stromal cell line M2-10B4 was plated in 96-well assay plates at a cell density of 1.0×10⁴ cells per well in 100 μL of growth medium. Assay plates were incubated at 37° C. in a 5% CO₂ incubator for 48 hours to form a confluent monolayer. Media was removed and cells were washed once in growth medium before plated on the stromal layer at a cell density of 5×10⁴ cells per well in 135 μL of growth medium in triplicate plates. The compound serial dilution was resuspended in 196 μL of RPMI-1640 and 15 μL of the dilution was added to the 135 μL containing the B and stromal cells. After incubating at 37° C. in a 5% CO₂ incubator for 18-24 hours, plates were centrifuged at 300×g for 5 minutes at room temperature. Seventy-five μL of supernatant from each well was used in the AlphaLISA detection system.

AlphaLISA Detection:

AlphaLISA assays were performed according to the manufacturer's instructions. The high sensitivity protocol was performed with 10 μL of supernatant from the assay wells. CCL3 and CCL4 release was detected with an Enspire AlphaLISA detection instrument (Perkin Elmer, Waltham, Mass.).

The MacSynergy II program was used to process the data and determine the inhibitory combination effect. The results are summarized in Table 6. The values of combination log volumes (μM². %) suggested that Compound B when combined with Compound D exhibited highly synergistic inhibitory effects in all tested cell lines. In addition, combination of Compound B and Compound D showed no antagonism in any of the cell lines tested.

TABLE 6
Inhibition of CCL3 and CCL4 by Compound B and Compound D in Combination

| | | Synergy/Antagonism volume [μM² · %] | | | | Combination |
|---|---|---|---|---|---|---|
| Cell Line | Chemokine | Type | Exp. 1 | Exp. 2 | Mean ± SD^{a} | effect |
| DHL-4 | CCL3 | Synergy | 980 | 1340 | 1160 ± 254 | Highly |
| | | Antagonism | 0 | −0.6 | | Synergistic |
| CCRF-SB | | Synergy | 118 | 295 | 206 ± 125 | Highly |
| | | Antagonism | 0 | 0 | | Synergistic |
| Mino | | Synergy | 351 | 823 | 587 ± 334 | Highly |
| | | Antagonism | 0 | 0 | | Synergistic |
| DHL-4 | CCL4 | Synergy | 1721 | 1304 | 1512.4 ± 294.7 | Highly |
| | | Antagonism | 0 | 0 | | Synergistic |
| CCRF-SB | | Synergy | 163 | 4833 | 323 ± 227 | Highly |
| | | Antagonism | −1.9 | 0 | | Synergistic |
| Mino | | Synergy | 412 | 731 | 571 ± 226 | Highly |
| | | Antagonism | 0 | 0 | | Synergistic |

^{a}Data represents mean values ± standard deviations from 2 independent experiments performed in triplicate.

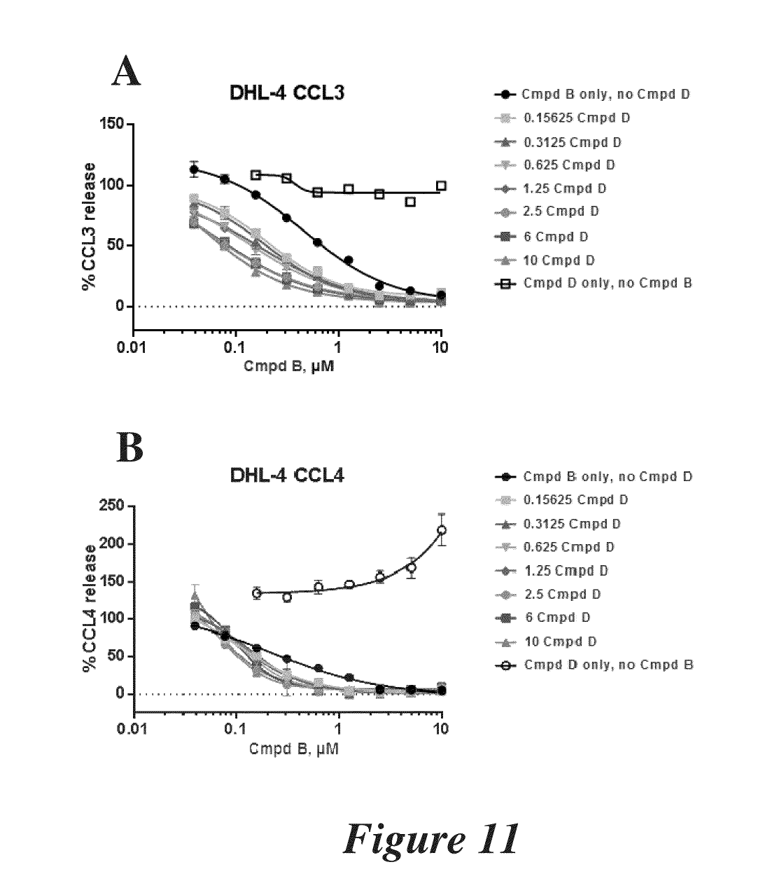

Inhibition curves from the combination drug treatment were generated for each cell line tested. A representative combination of Compound B and Compound D in inhibiting CCL3 and CCL4 production in DHL-4 are depicted in FIGS. 11A and 11B.

These results indicate that the combinations of Compound B and Compound D were highly synergistic in vitro inhibition of CCL3 and CCL4 release from CCRF-SB, DHL-4 and Mino cell lines that were co-cultured with a murine stromal cell line, M2-10B4. Synergy evaluations were based on statistical analysis using a Bonferroni adjusted log volume from 2 independent experiments. The synergy was similar for CCL4 and CCL3 in all 3 cell lines tested. The highest synergistic effect was detected for the drug combination in the DHL4 cell line and resulted in a CCL4 mean Bonferroni adjusted log volume of 1512.4±294.7 μM². % (n=2). The lowest synergistic effect was seen for CCL3 production in the CCRF-SB cells, where the mean Bonferroni adjusted log volume was 206.1±125 μM². % (n=2). Also, no antagonism between Compound D and Compound B was observed.

### Example 15

### Inhibition of Phospho-Mek and Phospho-Erk in B-Cell Lymphoma Cell Line by Compound B in Combination with Compound D

This Example demonstrates the in vitro activity of Compound B in combination with Compound D in inhibiting anti-IgG (αIgG)-stimulated phosphorylation of phospho-Mek (pMek) and phospho-Erk (pErk) expression in the diffuse large B-cell lymphoma cell line, DHL-4.

Compounds:

The compound samples were prepared according to the procedure described in Example 14 above.

Cell Lines:

SU-DHL-4 cell line was obtained from DSMZ (Leibniz-Institut DSMZ—Deutsche Sammlung von Mikroorganismen and Zellkulturen GmbH). DHL-4 were maintained in RPMI-1640 base medium containing 10% FBS, 1% penicillin-streptomycin, 10 mM HEPES and 1% GlutaMax.

Combination Assay:

pMek and pErk levels were determined using Meso Scale assays (Meso Scale Discovery, Rockville, Md.). Two-fold serial dilutions in 100% DMSO of Compound D, starting at 10 mM, were combined with 2-fold serial dilutions in 100% DMSO of Compound B, starting at 10 mM. DMSO containing αIgG at 10 μg/mL) was used as the 100% pMek or pErk signal control and DMSO containing no stimulation was used as the 0% pMek or pErk signal control.

Meso Scale Assays:

DHL-4 cells grown in log phase were starved in RPMI-1640 base medium containing 1% penicillin-streptomycin and 10 mM HEPES at 6×10⁶ cells/mL for 1 hour at 37° C. and 5% CO₂. Cells were seeded at 5×10⁵ cells per well in 96-well assay plates with fresh medium (RPMI-1640). Four μL of compound serial dilutions was resuspended in 196 μL of RPMI-1640. before added to the assay plate as a further 1:10 dilution. Compound was added to the cells and incubated for 1 hour at 37° C. and 5% CO₂. Cells were stimulated for 5 minutes with αIgG (10 μg/mL), except the 0% control cells, and centrifuged at 300×g for 5 minutes at room temperature. Supernatants were discarded, and cell pellets were lysed in 50 μL 1×RIPA buffer on ice for 10 minutes. Lysates were assayed in Meso Scale (MSD) plates or frozen at −80° C. until use. The pErk and pMek MSD assays were performed as per manufacturer's instructions. The plate was immediately read on the Meso Scale Sector 2400 Imager (Meso Scale Discovery, Gaithersburg, Md.). Data was normalized per well.

The MacSynergy II program was used to process the data and determine the inhibitory combination effect. The values of combination volumes (μM². %) showed that Compound B exhibited highly synergistic inhibitory effects when combined with Compound D. Table 7 summarized the mean Bonferroni adjusted log volumes of 279.4±211.1 μM². % and 135.5±29.2 μM². % for pErk and pMek respectively, from 2 individual experiments. Compound B and Compound D showed no sign of antagonism in any sample tested.

TABLE 7
Inhibition of αIgG-Mediated pMek and pErk in DHL-4
Cells by Compound B and Compound D in Combination

| | Synergy/Antagonism volume | | | | |
|---|---|---|---|---|---|
| | [μM² · %] | | | | Combination |
| Readout | Type | Exp. 1 | Exp. 2 | Mean ± SD^{a} | effect |
| pMek | Synergy | 156 | 115 | 136 ± 29 | Highly |
| | Antagonism | 0 | 0 | | Synergistic |
| pErk | Synergy | 429 | 130 | 279 + 211 | Highly |
| | Antagonism | 0 | −5.8 | | Synergistic |

^{a}Data represents mean values ± standard deviations from 2 independent experiments performed in triplicate.

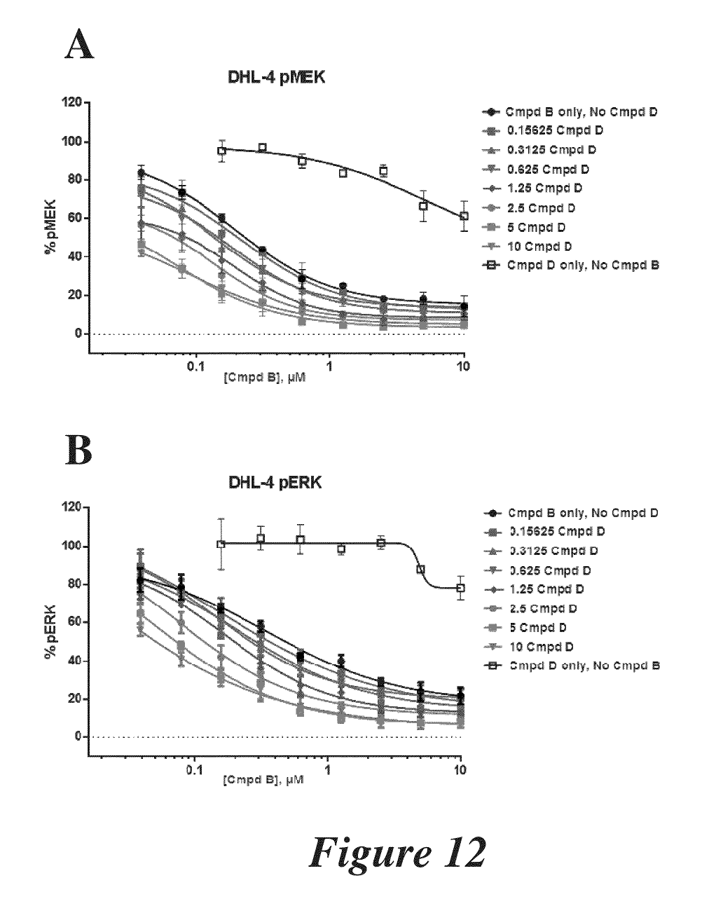

Inhibition curves from the combination treatment were generated for pMek and pErk in DHL-4 cells. A representative combination pMek and pERK curve dataset is depicted in FIGS. 12A and 12B.

This Example demonstrates that the combination of Compound B and Compound D exhibited highly synergistic in vitro inhibition of pMek and pErk expression in DHL-4 cells following αIgG stimulation of the BCR, as shown by the statistical analyses in this Example. No antagonism between Compound B and Compound D was observed.

What is claimed is:

1. A method for treating a human, who has or is suspected of having a cancer, comprising administering to the human an effective amount of Compound A 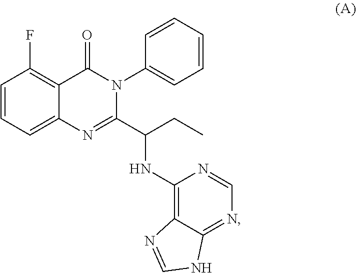 [SMILES: CCC(Nc1ncnc2[nH]cnc12)c1nc2cccc(F)c2c(=O)n1-c1ccccc1]
or a pharmaceutically acceptable salt thereof, and an effective amount of Compound B 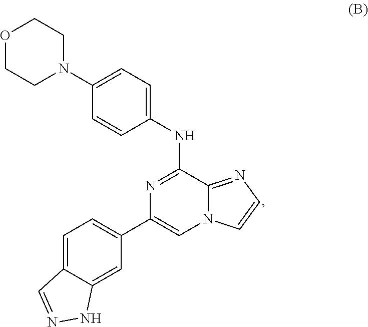 [SMILES: c1cn2cc(-c3ccc4cn[nH]c4c3)nc(Nc3ccc(N4CCOCC4)cc3)c2n1]
or a pharmaceutically acceptable salt thereof.

2. The method of claim 1, wherein Compound A or a pharmaceutically acceptable salt thereof is predominantly the (S)-enantiomer.

3. The method of claim 1, wherein:
Compound A or a pharmaceutically acceptable salt thereof is present in a pharmaceutical composition comprising Compound A or a pharmaceutically acceptable salt thereof, and at least one pharmaceutically acceptable vehicle; and
Compound B or a pharmaceutically acceptable salt thereof is present in a pharmaceutical composition comprising Compound B or a pharmaceutically acceptable salt thereof, and at least one pharmaceutically acceptable vehicle.

4. The method of claim 1, wherein the human who has cancer is (i) refractory to at least one chemotherapy treatment, or (ii) is in relapse after treatment with chemotherapy, or a combination thereof.

5. The method of claim 1, wherein the human has not previously been treated for the cancer.

6. The method of claim 1, wherein the cancer is leukemia, lymphoma, or multiple myeloma.

7. The method of claim 1, wherein the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL).

8. A kit comprising:
(i) a pharmaceutical composition comprising Compound A 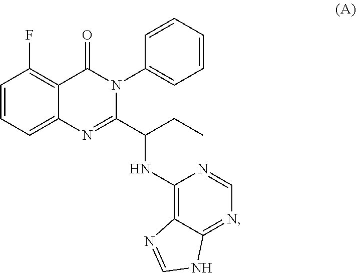 [SMILES: CCC(Nc1ncnc2[nH]cnc12)c1nc2cccc(F)c2c(=O)n1-c1ccccc1]
or a pharmaceutically acceptable salt thereof, and at least one pharmaceutically acceptable vehicle; and
(ii) a pharmaceutical composition comprising Compound B 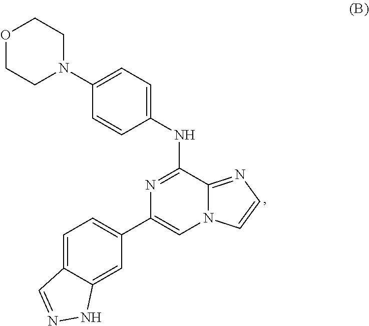 [SMILES: c1cn2cc(-c3ccc4cn[nH]c4c3)nc(Nc3ccc(N4CCOCC4)cc3)c2n1]
or a pharmaceutically acceptable salt thereof, and at least one pharmaceutically acceptable vehicle.

9. The kit of claim 8, further comprising: a package insert containing instructions for use of the pharmaceutical compositions in treating a cancer.

10. The kit of claim 8, wherein each pharmaceutical composition is independently a tablet.

11. The kit of claim 8, wherein the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL).

12. An article of manufacture comprising:
(i) a unit dosage form of Compound A 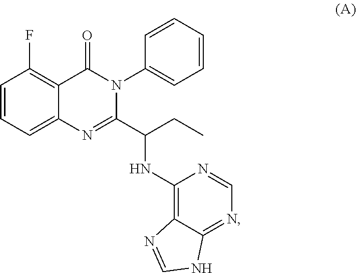 [SMILES: CCC(Nc1ncnc2[nH]cnc12)c1nc2cccc(F)c2c(=O)n1-c1ccccc1]
or a pharmaceutically acceptable salt thereof, and at least one pharmaceutically acceptable vehicle;
(ii) a unit dosage form of Compound B 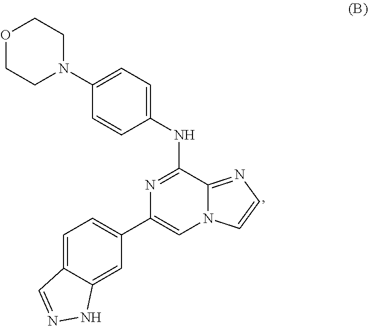 [SMILES: c1cn2cc(-c3ccc4cn[nH]c4c3)nc(Nc3ccc(N4CCOCC4)cc3)c2n1]
or a pharmaceutically acceptable salt thereof; and at least one pharmaceutically acceptable vehicle; and
(iii) a label containing instructions for use of Compound A and Compound B, or pharmaceutically acceptable salts thereof, in treating cancer.

13. The article of manufacture of claim 12, wherein each unit dosage form is a tablet.

14. The article of manufacture of claim 12, wherein the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL).
